(19) United States
(12) Patent Application Publication
(10) Pub. No.: US 2020/0105374 A1
(43) Pub. Date: Apr. 2, 2020

(54) MIXTURE MODEL FOR TARGETED SEQUENCING

(71) Applicant: Grail, Inc., Menlo Park, CA (US)

(72) Inventors: EARL HUBBELL, Palo Alto, CA (US); ARCHANA S. SHENOY, Menlo Park, CA (US)

(21) Appl. No.: 16/579,805

(22) Filed: Sep. 23, 2019

Related U.S. Application Data
(60) Provisional application No. 62/738,965, filed on Sep. 28, 2018.

(51) Int. Cl. G16B 30/10; G16B 20/00; G16B 45/00; G16B 40/00; G06N 3/08

(52) U.S. Cl. CPC G16B 30/10; G16B 20/00; G06N 3/08; G16B 40/00; G16B 45/00

(57) ABSTRACT

Systems and methods for determining a source of a variant in a cell free nucleic acid sample include identifying a candidate variant in the cell free nucleic acid sample, determining a numerical score using a measure of first properties of a distribution of novel somatic mutations compared to a measure of second properties of a distribution of somatic variants matched in genomic nucleic acid, and determining a classification of the candidate variant using the numerical score, the classification indicating whether the candidate variant is more likely to be a new novel somatic mutation than a new somatic variant matched in genomic nucleic acid.

### CROSS-REFERENCE TO RELATED APPLICATIONS

This application claims the benefit of priority to U.S. Provisional Patent Application No. 62/738,965, filed on Sep. 28, 2018, and entitled “MIXTURE MODEL FOR TARGETED SEQUENCING,” the contents of which are herein incorporated by reference in their entirety.

### BACKGROUND

### 1. Field of Art

This disclosure generally relates to targeted sequencing and more specifically to using a model for predicting sources of variants.

### 2. Introduction

Analysis of circulating cell free nucleotides, such as cell free DNA (cfDNA) or cell free RNA (cfRNA), using next generation sequencing (NGS) is recognized as a valuable tool for detection and diagnosis of cancer or other diseases. Identifying rare variants indicative of cancer using NGS requires deep sequencing of nucleotide sequences from a biological sample such as a tissue biopsy or blood drawn from a subject. Detecting DNA that originated from tumor cells from a blood sample is difficult because circulating tumor DNA (ctDNA) is typically present at low levels relative to other molecules in cfDNA extracted from the blood. The inability of existing methods to identify true positives (e.g., indicative of cancer in the subject) from signal noise diminishes the ability of known and future systems to distinguish true positives from false positives caused by noise sources, which can result in unreliable results for variant calling or other types of analyses. Moreover, errors introduced during sample preparation and sequencing can make accurate identification of rare variants difficult.

A number of different methods have been developed for detecting variants, such as single nucleotide variants (SNVs), in sequencing data. Most conventional methods have been developed for calling variants from DNA sequencing data obtained from a tissue sample. These methods may not be suitable for calling variants from deep sequencing data obtained from a cell free nucleotide sample.

For non-invasive diagnostic and monitoring of cancer, targeted sequencing data of cell free nucleotides serve as an important bio-source. However, detection of variants in deep sequencing data sets poses distinct challenges: the number of sequenced fragments tend to be several order of magnitude larger (e.g., sequencing depth can be 2,000× or more), debilitating most of the existing variant callers in compute-time and memory usage.

A major challenge to accurate detection of variants is the possibility of damage to sequenced fragments that occur during processing. An example of damage to sequenced fragments can be a nucleotide substitution that occurs naturally or due to assay processing steps. For example, damage can occur due to spontaneous deamination of nucleotide bases or due to end repair errors. Since the damage occurs during processing, existing variant callers may identify these nucleotide base changes as variants in the genome. In other words, this damage can lead to systematic errors and can cause mutations to be falsely identified, e.g., as false positives.

### SUMMARY

In various embodiments, a method for determining a source of a variant in a cell free nucleic acid sample comprises identifying a candidate variant in the cell free nucleic acid sample. The method further comprises determining a numerical score using a measure of first properties of a distribution of novel somatic mutations compared to a measure of second properties of a distribution of somatic variants matched in genomic nucleic acid. The method further comprises determining a classification of the candidate variant using the numerical score, the classification indicating whether the candidate variant is more likely to be a new novel somatic mutation than a new somatic variant matched in genomic nucleic acid.

In some embodiments, the first properties of the distribution of novel somatic mutations and the second properties of the distribution of somatic variants matched in genomic nucleic acid are modeled by generalized linear models.

In some embodiments, the generalized linear models each generate outcomes from a gamma distribution. In some embodiments, the generalized linear models each generate outcomes from a normal distribution, binomial distribution, or Poisson distribution.

In some embodiments, the generalized linear models are trained by modeling a true alternate frequency of the candidate variant in a given genomic nucleic acid sample as dependent on a true alternate frequency of the candidate variant in a given cell free nucleic acid sample.

In some embodiments, the numerical score is determined at least by modeling alternate allele counts of the candidate variant using a Poisson distribution after a gamma distribution.

In some embodiments, the measure of first properties or the measure of second properties represents a likelihood under a generalized linear model using a gamma distribution with Poisson counts.

In some embodiments, the method further comprises adjusting the numerical score by modifying the likelihood under the generalized linear model by an empirical adjustment factor.

In some embodiments, the first and second properties include one or more of depth, alternate frequency, or trinucleotide context of a given nucleic acid sample.

In some embodiments, the numerical score is further determined by comparing the first properties, the second properties, and third properties of a distribution of variants associated with a source different from the novel somatic mutations and the somatic variants matched in genomic nucleic acid.

In some embodiments, the somatic variants matched in genomic nucleic acid are matched with variants observed in white blood cells.

In some embodiments, determining the numerical score comprises determining a first likelihood l_{NS} of observing the novel somatic mutations based on an alternate frequency of the novel somatic mutations.

In some embodiments, the method further comprises determining that the candidate variant is located on a certain gene; and determining a second likelihood π_{NS,gene} that the certain gene will have at least one mutation based on observed data from training samples of the certain gene. The numerical score is determined based at least in part on a product of the first likelihood and the second likelihood.

In some embodiments, the method further comprises determining an attribute of an individual from whom the cell free nucleic acid sample was obtained; and determining a third likelihood π_{NS,person} that the individual will have the candidate variant based on observed data from training samples of individuals associated with the attribute, the product further including the third likelihood. In some embodiments, the attribute is an age or an age range.

In some embodiments, determining the classification of the candidate variant comprises determining an integral of a plurality of negative binomial distributions over an expected distribution of alternate frequency of the candidate variant in a given cell free nucleic acid sample.

In some embodiments, the plurality of negative binomial distributions model expected distributions of false positive and true positive mutations of the candidate variant in a given cell free nucleic acid sample.

In some embodiments, the plurality of negative binomial distributions account for depths of sequence reads of samples of the novel somatic mutations and the somatic variants matched in genomic nucleic acid.

In some embodiments, the integral is calculated numerically using a trapezoidal sum.

In some embodiments, the somatic variants matched in genomic nucleic acid are associated with clonal hematopoiesis.

In some embodiments, the method further comprises obtaining the cell free nucleic acid sample from an individual; labeling fragments in the cell free nucleic acid sample; performing enrichment on the cell free nucleic acid sample to amplify the labeled fragments; and generating a plurality of sequence reads using the enriched cell free nucleic acid sample, the candidate variant identified from the plurality of sequence reads.

In some embodiments, the method further comprises determining a prediction that the candidate variant is a true mutation in the cell free nucleic acid sample based on the classification; and determining a likelihood that an individual has a disease based at least in part on the prediction.

In various embodiments, a method for modeling sources of variants in nucleic acid samples comprises obtaining a first set of training samples of novel somatic mutations. The method further comprises obtaining a second set of training samples of somatic variants matched in genomic nucleic acid. The method further comprises determining a first shape parameter and a first slope parameter of a first generalized linear model by iteratively modeling variance of the first set of training samples as a first gamma distribution. The method further comprises determining a second shape parameter and a second slope parameter of a second generalized linear model by iteratively modeling variance of the second set of training samples as a second gamma distribution. The method further comprises storing the first and second shape parameters and the first and second slope parameters, the stored parameters used for determining whether a candidate variant is more likely to be a novel somatic mutation than a somatic variant matched in genomic nucleic acid.

In some embodiments, iteratively modeling variance of the first and second sets of training samples comprises modifying at least one of the first and second sets of training samples using samples of individuals known to have a certain disease or not; determining updated parameters for the first and second generalized linear models using the modified at least one training sample; and determining pairs of precision and recall values for predicting true mutations of a test set of cell free nucleic acid samples using the updated parameters.

In some embodiments, the method further comprises determining a precision value and a corresponding recall value of one of the pairs of precision and recall values; determining that the precision value is greater than a threshold precision; and determining that the recall value is greater than a threshold recall.

In some embodiments, the updated parameters are iteratively determined using an expectation-maximization algorithm.

In some embodiments, the first shape parameter is greater than the second shape parameter.

In some embodiments, a system comprises a computer processor and a memory, the memory storing instructions that when executed by the computer processor cause the processor to carry out the steps of any of the preceding paragraphs.

### BRIEF DESCRIPTION OF DRAWINGS

FIG. 1A is flowchart of a method for preparing a nucleic acid sample for sequencing according to some embodiments.

FIG. 1B is a graphical representation of the process for obtaining sequence reads according to some embodiments.

FIG. 2 is block diagram of a processing system for processing sequence reads according to some embodiments.

FIG. 3 is flowchart of a method for determining variants of sequence reads according to some embodiments.

FIG. 4 is a diagram of an application of a Bayesian hierarchical model according to some embodiments.

FIG. 5A shows dependencies between parameters and sub-models of a Bayesian hierarchical model for determining true single nucleotide variants according to some embodiments.

FIG. 5B shows dependencies between parameters and sub-models of a Bayesian hierarchical model for determining true insertions or deletions according to some embodiments.

FIG. 6A shows an example diagram of noise rates associated with a Bayesian hierarchical model according to some embodiments.

FIG. 6B shows an example diagram of alternate depth (AD) associated with a Bayesian hierarchical model according to some embodiments.

FIG. 7A is a diagram of determining parameters by fitting a Bayesian hierarchical model according to some embodiments.

FIG. 7B is a diagram of using parameters from a Bayesian hierarchical model to determine a likelihood of a false positive according to some embodiments.

FIG. 8 is flowchart of a method for training a Bayesian hierarchical model according to some embodiments.

FIG. 9 is flowchart of a method for scoring candidate variants of a given nucleotide mutation according to some embodiments.

FIG. 10 is flowchart of a method for using a joint model to process cell free nucleic acid samples and genomic nucleic acid samples according to some embodiments.

FIG. 11 is a diagram of an application of a joint model according to some embodiments.

FIG. 12 is a diagram of observed counts of variants in samples from healthy individuals according to some embodiments.

FIG. 13 illustrates distributions of training data sets having different types of mutations according to some embodiments.

FIG. 14 is flowchart of a method for classifying candidate variants in nucleic acid samples according to some embodiments.

FIG. 15 is flowchart of a method for determining numerical scores for candidate variants according to some embodiments.

FIG. 16 is a diagram of shape parameters determined by modeling training data sets having different types of mutations according to some embodiments.

FIG. 17 is a diagram of slope parameters determined by modeling training data sets having different types of mutations according to some embodiments.

FIG. 18 is a diagram of counts of variants based on age of individuals according to some embodiments.

FIG. 19 is a diagram of labeled training data sets according to some embodiments.

FIG. 20 is another diagram of labeled training data sets according to some embodiments.

FIG. 21 is a diagram of classified training data sets according to some embodiments.

FIG. 22 is a diagram of counts of single nucleotide variants and insertions or deletions sets according to some embodiments.

FIG. 23 illustrates a precision recall curve of a model according to some embodiments.

FIG. 24 is flowchart of a method for tuning a mixture model according to some embodiments.

FIG. 25 illustrates distributions of variance for clonal hematopoiesis and novel somatic variants according to some embodiments.

FIG. 26 illustrates histograms of draws from posterior distributions for parameters of distributions of variant calls according to some embodiments.

FIG. 27 illustrates another precision recall curve of a tuned model according to some embodiments.

FIG. 28 illustrates detected outliers of a distribution of training data sets according to some embodiments.

FIG. 29 shows a schematic of an example computer system for implementing various methods of the present invention.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

Reference will now be made in detail to several embodiments, examples of which are illustrated in the accompanying figures. It is noted that wherever practicable similar or like reference numbers may be used in the figures and may indicate similar or like functionality. For example, a letter after a reference numeral, such as “sequence reads 180A,” indicates that the text refers specifically to the element having that particular reference numeral. A reference numeral in the text without a following letter, such as “sequence reads 180,” refers to any or all of the elements in the figures bearing that reference numeral (e.g. “sequence reads 180” in the text refers to reference numerals “sequence reads 180A” and/or “sequence reads 180B” in the figures).

### I. Definitions

The term “individual” refers to a human individual. The term “healthy individual” refers to an individual presumed to not have a cancer or disease. The term “subject” refers to an individual who is known to have, or potentially has, a cancer or disease.

The term “sequence reads” refers to nucleotide sequences read from a sample obtained from an individual. Sequence reads can be obtained through various methods known in the art.

The term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

The term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

The term “indel” refers to any insertion or deletion of one or more bases having a length and a position (which can also be referred to as an anchor position) in a sequence read. An insertion corresponds to a positive length, while a deletion corresponds to a negative length.

The term “mutation” refers to one or more SNVs or indels.

The term “true positive” refers to a mutation that indicates real biology, for example, presence of a potential cancer, disease, or germline mutation in an individual. True positives are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive. Generally, false positives can be more likely to occur when processing sequence reads associated with greater mean noise rates or greater uncertainty in noise rates.

The term “cell free nucleic acid,” “cell free DNA,” or “cfDNA” refers to nucleic acid fragments that circulate in an individual's body (e.g., bloodstream) and originate from one or more healthy cells and/or from one or more cancer cells.

The term “circulating tumor DNA” or “ctDNA” refers to deoxyribonucleic acid fragments that originate from tumor cells or other types of cancer cells, which may be released into an individual's bloodstream as result of biological processes such as apoptosis or necrosis of dying cells or actively released by viable tumor cells.

The term “genomic nucleic acid,” “genomic DNA,” or “gDNA” refers to nucleic acid including chromosomal DNA that originate from one or more healthy cells.

The term “alternative allele,” “alternate allele,” or “ALT” refers to an allele having one or more mutations relative to a reference allele, e.g., corresponding to a known gene.

The term “sequencing depth” or “depth” refers to a total number of read segments from a sample obtained from an individual.

The term “alternate depth” or “AD” refers to a number of read segments in a sample that support an ALT, e.g., include mutations of the ALT.

The term “reference depth” refers to a number of read segments in a sample that include a reference allele at a candidate variant location.

The term “alternate frequency,” “allele frequency,” or “AF” refers to the frequency of a given ALT. The AF may be determined by dividing the corresponding AD of a sample by the depth of the sample for the given ALT.

The term “variant” or “true variant” refers to a mutated nucleotide base at a position in the genome. Such a variant can lead to the development and/or progression of cancer in an individual.

The term “edge variant” refers to a mutation located near an edge of a sequence read, for example, within a threshold distance of nucleotide bases from the edge of the sequence read.

The term “candidate variant,” “called variant,” “putative variant,” or refers to one or more detected nucleotide variants of a nucleotide sequence, for example, at a position in the genome that is determined to be mutated. Generally, a nucleotide base is deemed a called variant based on the presence of an alternative allele on sequence reads obtained from a sample, where the sequence reads each cross over the position in the genome. The source of a candidate variant can initially be unknown or uncertain. During processing, candidate variants can be associated with an expected source such as gDNA (e.g., blood-derived) or cells impacted by cancer (e.g., tumor-derived). Additionally, candidate variants can be called as true positives.

The term “non-edge variant” refers to a candidate variant that is not determined to be resulting from an artifact process, e.g., using an edge variant filtering method described herein. In some scenarios, a non-edge variant may not be a true variant (e.g., mutation in the genome) as the non-edge variant could arise due to a different reason as opposed to one or more artifact processes.

### II. Example Assay Protocol

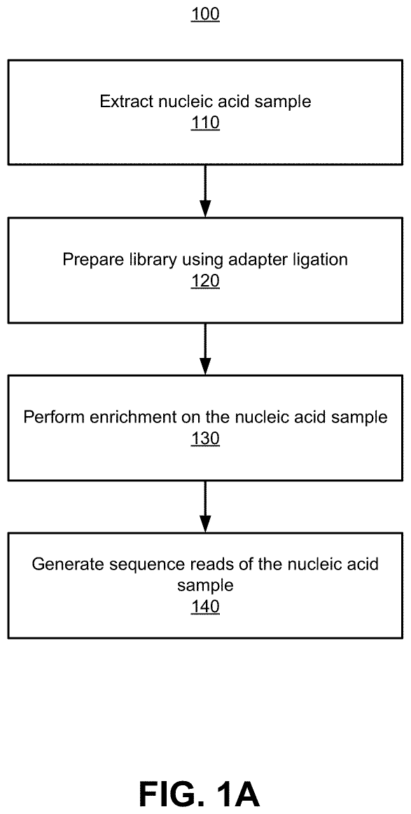

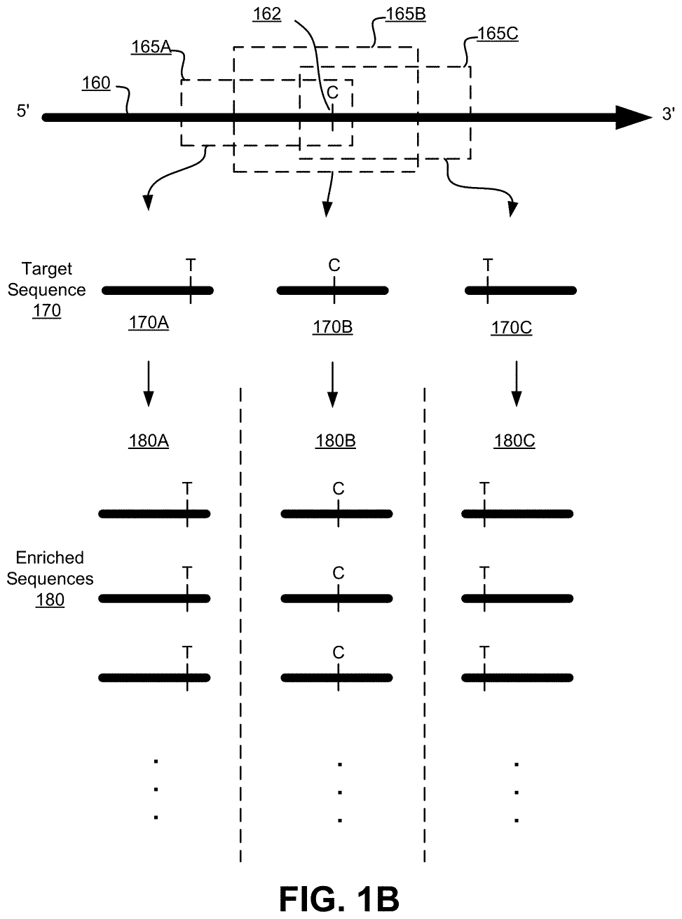

FIG. 1A is flowchart of a method 100 for preparing a nucleic acid sample for sequencing according to some embodiments. The method 100 includes, but is not limited to, the following steps. For example, any step of the method 100 can comprise a quantitation sub-step for quality control or other laboratory assay procedures known to one skilled in the art.

In step 110, a nucleic acid sample (DNA or RNA) is extracted from a subject. In the present disclosure, DNA and RNA can be used interchangeably unless otherwise indicated. That is, the following embodiments for using error source information in variant calling and quality control can be applicable to both DNA and RNA types of nucleic acid sequences. However, some examples described herein focus on DNA for purposes of clarity and explanation. The sample can be any subset of the human genome, including the whole genome. The sample can be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) can be less invasive than procedures for obtaining a tissue biopsy, which can require surgery. The extracted sample can comprise cfDNA and/or ctDNA. For healthy individuals, the human body can naturally clear out cfDNA and other cellular debris. If a subject has a cancer or disease, ctDNA in an extracted sample can be present at a detectable level for diagnosis.

In step 120, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In step 130, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer type or tissue of origin). For a given workflow, the probes can be designed to anneal (or hybridize) to a target (complementary) strand of DNA or RNA. The target strand can be the “positive” strand (e.g., the strand transcribed into mRNA, and subsequently translated into a protein) or the complementary “negative” strand. The probes can range in length from 10s, 100s, or 1000s of base pairs. In some embodiments, the probes are designed based on a gene panel to analyze particular mutations or target regions of the genome (e.g., of the human or another organism) that are suspected to correspond to certain cancers or other types of diseases. Moreover, the probes can cover overlapping portions of a target region.

FIG. 1B is a graphical representation of the process for obtaining sequence reads according to some embodiments. FIG. 1B depicts one example of a nucleic acid segment 160 from the sample. Here, the nucleic acid segment 160 can be a single-stranded nucleic acid segment, such as a single stranded DNA or single stranded RNA segment. In some embodiments, the nucleic acid segment 160 is a double-stranded cfDNA segment. The illustrated example depicts three regions 165A, 165B, and 165C of the nucleic acid segment 160 that can be targeted by different probes. Specifically, each of the three regions 165A, 165B, and 165C includes an overlapping position on the nucleic acid segment 160. An example overlapping position is depicted in FIG. 1B as the cytosine (“C”) nucleotide base 162. The cytosine nucleotide base 162 is located near a first edge of region 165A, at the center of region 165B, and near a second edge of region 165C.

In some embodiments, one or more (or all) of the probes are designed based on a gene panel to analyze particular mutations or target regions of the genome (e.g., of the human or another organism) that are suspected to correspond to certain cancers or other types of diseases. By using a targeted gene panel rather than sequencing all expressed genes of a genome, also known as “whole exome sequencing,” the method 100 can be used to increase sequencing depth of the target regions, where depth refers to the count of the number of times a given target sequence within the sample has been sequenced. Increasing sequencing depth reduces required input amounts of the nucleic acid sample.

Hybridization of the nucleic acid sample 160 using one or more probes results in an understanding of a target sequence 170. As shown in FIG. 1B, the target sequence 170 is the nucleotide base sequence of the region 165 that is targeted by a hybridization probe. The target sequence 170 can also be referred to as a hybridized nucleic acid fragment. For example, target sequence 170A corresponds to region 165A targeted by a first hybridization probe, target sequence 170B corresponds to region 165B targeted by a second hybridization probe, and target sequence 170C corresponds to region 165C targeted by a third hybridization probe. Given that the cytosine nucleotide base 162 is located at different locations within each region 165A-C targeted by a hybridization probe, each target sequence 170 includes a nucleotide base that corresponds to the cytosine nucleotide base 162 at a particular location on the target sequence 170.

In the example of FIG. 1B, the target sequence 170A and target sequence 170C each have a nucleotide base (shown as thymine “T”) that is located near the edge of the target sequences 170A and 170C. Here, the thymine nucleotide base (e.g., as opposed to a cytosine base) can be a result of a random cytosine deamination process that causes a cytosine base to be subsequently recognized as a thymine nucleotide base during the sequencing process. Thus, the C>T SNV for target sequences 170A and 170C can be considered an edge variant because the mutation is located at an edge of target sequences 170A and 170C. A cytosine deamination process can lead to a downstream sequencing artifact that prevents the accurate capture of the actual nucleotide base pair in the nucleic acid segment 160. Additionally, target sequence 170B has a cytosine base that is located at the center of the target sequence 170B. Here, a cytosine base that is located at the center can be less susceptible to cytosine deamination.

After a hybridization step, the hybridized nucleic acid fragments are captured and can also be amplified using PCR. For example, the target sequences 170 can be enriched to obtain enriched sequences 180 that can be subsequently sequenced. In some embodiments, each enriched sequence 180 is replicated from a target sequence 170. Enriched sequences 180A and 180C that are amplified from target sequences 170A and 170C, respectively, also include the thymine nucleotide base located near the edge of each sequence read 180A or 180C. As used hereafter, the mutated nucleotide base (e.g., thymine nucleotide base) in the enriched sequence 180 that is mutated in relation to the reference allele (e.g., cytosine nucleotide base 162) is considered as the alternative allele. Additionally, each enriched sequence 180B amplified from target sequence 170B includes the cytosine nucleotide base located near or at the center of each enriched sequence 180B.

In step 140, sequence reads are generated from the enriched DNA sequences, e.g., enriched sequences 180 shown in FIG. 1B. Sequencing data can be acquired from the enriched DNA sequences by known means in the art. For example, the method 100 can include next generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads can be aligned to a reference genome using known methods in the art to determine alignment position information. The alignment position information can indicate a beginning position and an end position of a region in the reference genome that corresponds to a beginning nucleotide base and end nucleotide base of a given sequence read. Alignment position information can also include sequence read length, which can be determined from the beginning position and end position. A region in the reference genome can be associated with a gene or a segment of a gene.

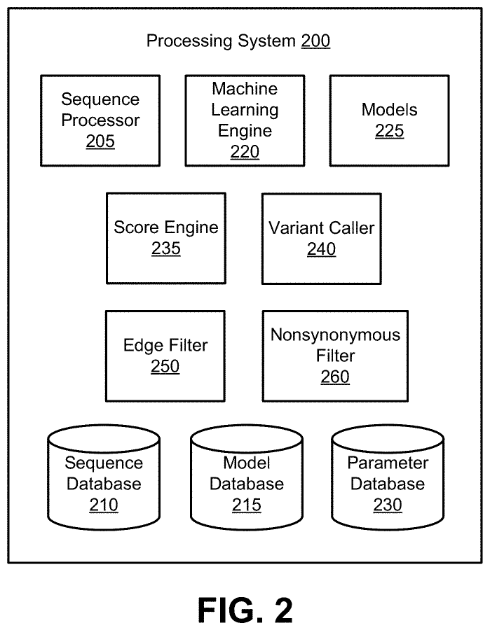

In various embodiments, a sequence read is comprised of a read pair denoted as R₁ and R₂. For example, the first read R₁ can be sequenced from a first end of a nucleic acid fragment whereas the second read R₂ can be sequenced from the second end of the nucleic acid fragment. Therefore, nucleotide base pairs of the first read R₁ and second read R₂ can be aligned consistently (e.g., in opposite orientations) with nucleotide bases of the reference genome. Alignment position information derived from the read pair R₁ and R₂ can include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format can be generated and output for further analysis such as variant calling, as described below with respect to FIG. 2.

In an example process performed for a targeted sequencing assay, two tubes of whole blood were drawn into Streck BCTs from healthy individuals (self-reported as no cancer diagnosis). After plasma was separated from the whole blood, it was stored at −80° C. Upon assay processing, cfDNA was extracted and pooled from two tubes of plasma. Corielle genomic DNA (gDNA) were fragmented to a mean size of 180 base pairs (bp) and then sized selected to a tighter distribution using magnetic beads. The library preparation protocol was optimized for low input cell free DNA (cfDNA) and sheared gDNA. Unique molecular identifiers (UMIs) were incorporated into the DNA molecules during adapter ligation. Flowcell clustering adapter sequences and dual sample indices were then incorporated at library preparation amplification with PCR. Libraries were enriched using a targeted capture panel.

Target DNA molecules were first captured using biotinlyated single-stranded DNA hybridization probes and then enriched using magnetic streptavidin beads. Non-target molecules were removed using subsequent wash steps. The HiSeq X Reagent Kit v2.5 (Illumina; San Diego, Calif.) was used for flowcell clustering and sequencing. Four libraries per flowcell were multiplexed. Dual indexing primer mix was included to enable dual sample indexing reads. The read lengths were set to 150, 150, 8, and 8, respectively for read 1, read 2, index read 1, and index read 2. The first 6 base reads in read 1 and read 2 are the UMI sequences.

### III. Example Processing System

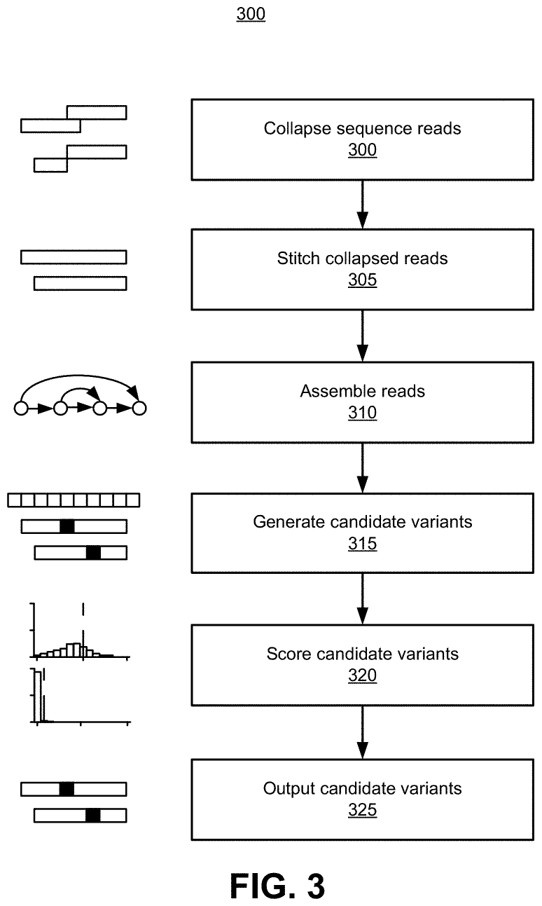

FIG. 2 is block diagram of a processing system 200 for processing sequence reads according to some embodiments. The processing system 200 includes a sequence processor 205, sequence database 210, model database 215, machine learning engine 220, models 225 (for example, including one or more Bayesian hierarchical models, joint models, or mixture models), parameter database 230, score engine 235, variant caller 240, edge filter 250, and non-synonymous filter 260. FIG. 3 is flowchart of a method 300 for determining variants of sequence reads according to some embodiments. In some embodiments, the processing system 200 performs the method 300 to perform variant calling (e.g., for SNVs and/or indels) based on input sequencing data. Further, the processing system 200 can obtain the input sequencing data from an output file associated with nucleic acid sample prepared using the method 100 described above. The method 300 includes, but is not limited to, the following steps, which are described with respect to the components of the processing system 200. In other embodiments, one or more steps of the method 300 can be replaced by a step of a different process for generating variant calls, e.g., using Variant Call Format (VCF), such as HaplotypeCaller, VarScan, Strelka, or SomaticSniper.

At step 300, the sequence processor 205 collapses aligned sequence reads of the input sequencing data. In some embodiments, collapsing sequence reads includes using UMIs, and optionally alignment position information from sequencing data of an output file (e.g., from the method 100 shown in FIG. 1) to collapse multiple sequence reads into a consensus sequence for determining the most likely sequence of a nucleic acid fragment or a portion thereof. The unique sequence tag can be from about 4 to 20 nucleic acids in length. Since the UMIs are replicated with the ligated nucleic acid fragments through enrichment and PCR, the sequence processor 205 can determine that certain sequence reads originated from the same molecule in a nucleic acid sample. In some embodiments, sequence reads that have the same or similar alignment position information (e.g., beginning and end positions within a threshold offset) and include a common UMI are collapsed, and the sequence processor 205 generates a collapsed read (also referred to herein as a consensus read) to represent the nucleic acid fragment. The sequence processor 205 designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the sequence processor 205 can perform other types of error correction on sequence reads as an alternate to, or in addition to, collapsing sequence reads.

At step 305, the sequence processor 205 stitches the collapsed reads based on the corresponding alignment position information. In some embodiments, the sequence processor 205 compares alignment position information between a first read and a second read to determine whether nucleotide base pairs of the first and second reads overlap in the reference genome. In some use cases, responsive to determining that an overlap (e.g., of a given number of nucleotide bases) between the first and second reads is greater than a threshold length (e.g., threshold number of nucleotide bases), the sequence processor 205 designates the first and second reads as “stitched”; otherwise, the collapsed reads are designated “unstitched.” In some embodiments, a first and second read are stitched if the overlap is greater than the threshold length and if the overlap is not a sliding overlap. For example, a sliding overlap can include a homopolymer run (e.g., a single repeating nucleotide base), a dinucleotide run (e.g., two-nucleotide base sequence), or a trinucleotide run (e.g., three-nucleotide base sequence), where the homopolymer run, dinucleotide run, or trinucleotide run has at least a threshold length of base pairs.

At step 310, the sequence processor 205 assembles reads into paths. In some embodiments, the sequence processor 205 assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The sequence processor 205 aligns collapsed reads to a directed graph such that any of the collapsed reads can be represented in order by a subset of the edges and corresponding vertices.

In some embodiments, the sequence processor 205 determines sets of parameters describing directed graphs and processes directed graphs. Additionally, the set of parameters can include a count of successfully aligned k-mers from collapsed reads to a k-mer represented by a node or edge in the directed graph. The sequence processor 205 stores, e.g., in the sequence database 210, directed graphs and corresponding sets of parameters, which can be retrieved to update graphs or generate new graphs. For instance, the sequence processor 205 can generate a compressed version of a directed graph (e.g., or modify an existing graph) based on the set of parameters. In some use cases, in order to filter out data of a directed graph having lower levels of importance, the sequence processor 205 removes (e.g., “trims” or “prunes”) nodes or edges having a count less than a threshold value, and maintains nodes or edges having counts greater than or equal to the threshold value.

At step 315, the variant caller 240 generates candidate variants from the paths assembled by the sequence processor 205. In some embodiments, the variant caller 240 generates the candidate variants by comparing a directed graph (which can be compressed by pruning edges or nodes in step 310) to a reference sequence of a target region of a genome. The variant caller 240 can align edges of the directed graph to the reference sequence, and record the genomic positions of mismatched edges and mismatched nucleotide bases adjacent to the edges as the locations of candidate variants. In some embodiments, the genomic positions of mismatched edges and mismatched nucleotide bases to the left and right of edges are recorded as the locations of called variants. Additionally, the variant caller 240 can generate candidate variants based on the sequencing depth of a target region. In particular, the variant caller 240 can be more confident in identifying variants in target regions that have greater sequencing depth, for example, because a greater number of sequence reads help to resolve (e.g., using redundancies) mismatches or other base pair variations between sequences.

In some embodiments, the variant caller 240 generates candidate variants using a model 225 to determine expected noise rates for sequence reads from a subject. The model 225 can be a Bayesian hierarchical model, though in some embodiments, the processing system 200 uses one or more different types of models. Moreover, a Bayesian hierarchical model can be one of many possible model architectures that may be used to generate candidate variants and which are related to each other in that they all model position-specific noise information in order to improve the sensitivity/specificity of variant calling. More specifically, the machine learning engine 220 trains the model 225 using samples from healthy individuals to model the expected noise rates per position of sequence reads.

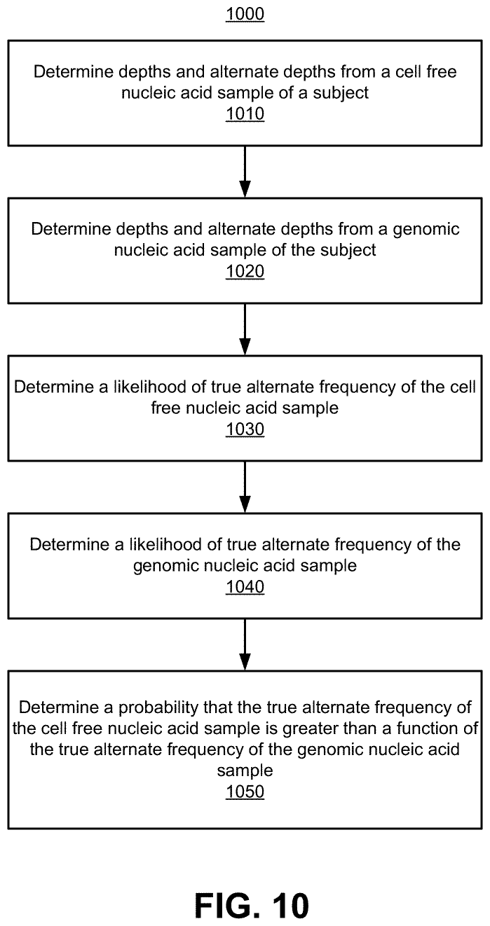

Further, multiple different models can be stored in the model database 215 or retrieved for application post-training. For example, a first model is trained to model SNV noise rates and a second model is trained to model indel noise rates. Further, the score engine 235 can use parameters of the model 225 to determine a likelihood of one or more true positives in a sequence read. The score engine 235 can determine a quality score (e.g., on a logarithmic scale) based on the likelihood. For example, the quality score is a Phred quality score Q=−10·log₁₀ P, where P is the likelihood of an incorrect candidate variant call (e.g., a false positive). Other models 225 such as a joint model (further described below with reference to FIGS. 10-12) can use output of one or more Bayesian hierarchical models to determine expected noise of nucleotide mutations in sequence reads of different samples.

At step 320, the processing system 200 filters the candidate variants using one or more types of models 225 or filters. For example, the score engine 235 scores the candidate variants using a joint model, edge variant prediction model, or corresponding likelihoods of true positives or quality scores. In addition, the processing system 200 can filter edge variants and/or non-synonymous mutations using the edge filter 250 and/or nonsynonymous filter 260, respectively.

At step 325, the processing system 200 outputs the filtered candidate variants. In some embodiments, the processing system 200 outputs some or all of the determined candidate variants along with corresponding one scores from the filtering steps. Downstream systems, e.g., external to the processing system 200 or other components of the processing system 200, can use the candidate variants and scores for various applications including, but not limited to, predicting presence of cancer, disease, or germline mutations.

### IV. Example Noise Models

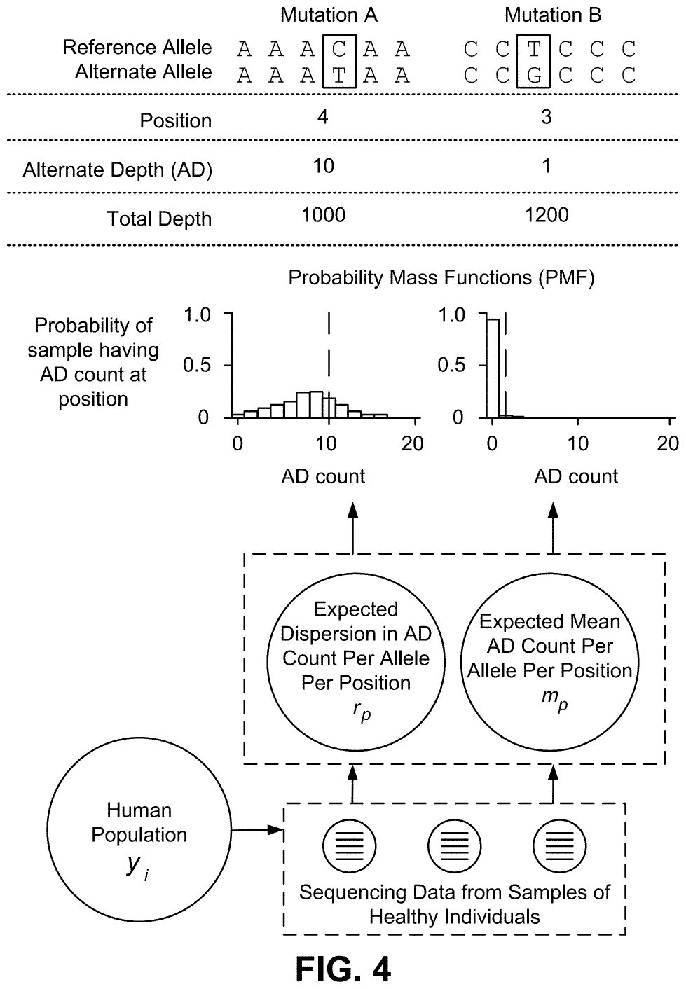

FIG. 4 is a diagram of an application of a Bayesian hierarchical model (e.g., model 225) according to some embodiments. Mutation A and Mutation B are shown as examples for purposes of explanation. In the embodiment of FIG. 4, Mutations A and B are represented as SNVs, though in other embodiments, the following description is also applicable to indels or other types of mutations. Mutation A is a C>T mutation at position 4 of a first reference allele from a first sample. The first sample has a first AD of 10 and a first total depth of 1000. Mutation B is a T>G mutation at position 3 of a second reference allele from a second sample. The second sample has a second AD of 1 and a second total depth of 1200. Based merely on AD (or AF), Mutation A can appear to be a true positive, while Mutation B can appear to be a false positive because the AD (or AF) of the former is greater than that of the latter. However, Mutations A and B can have different relative levels of noise rates per allele and/or per position of the allele. In fact, Mutation A can be a false positive and Mutation B can be a true positive, once the relative noise levels of these different positions are accounted for. The models 225 described herein model this noise for appropriate identification of true positives accordingly.

The probability mass functions (PMFs) illustrated in FIG. 4 indicate the probability (or likelihood) of a sample from a subject having a given AD count at a position. Using sequencing data from samples of healthy individuals (e.g., stored in the sequence database 210), the processing system 200 trains a model 225 from which the PDFs for healthy samples can be derived. In particular, the PDFs are based on mₚ, which models the expected mean AD count per allele per position in normal tissue (e.g., of a healthy individual), and rₚ, which models the expected variation (e.g., dispersion) in this AD count. Stated differently, mₚ and/or rₚ represent a baseline level of noise, on a per position per allele basis, in the sequencing data for normal tissue.

Using the example of FIG. 4 to further illustrate, samples from the healthy individuals represent a subset of the human population modeled by yᵢ, where i is the index of the healthy individual in the training set. Assuming for sake of example the model 225 has already been trained, PDFs produced by the model 225 visually illustrate the likelihood of the measured ADs for each mutation, and therefore provide an indication of which are true positives and which are false positives. The example PDF on the left of FIG. 4 associated with Mutation A indicates that the probability of the first sample having an AD count of 10 for the mutation at position 4 is approximately 20%. Additionally, the example PDF on the right associated with Mutation B indicates that the probability of the second sample having an AD count of 1 for the mutation at position 3 is approximately 1% (note: the PDFs of FIG. 4 are not exactly to scale). Thus, the noise rates corresponding to these probabilities of the PDFs indicate that Mutation A is more likely to occur than Mutation B, despite Mutation B having a lower AD and AF. Thus, in this example, Mutation B can be the true positive and Mutation A can be the false positive. Accordingly, the processing system 200 can perform improved variant calling by using the model 225 to distinguish true positives from false positives at a more accurate rate, and further provide numerical confidence as to these likelihoods.

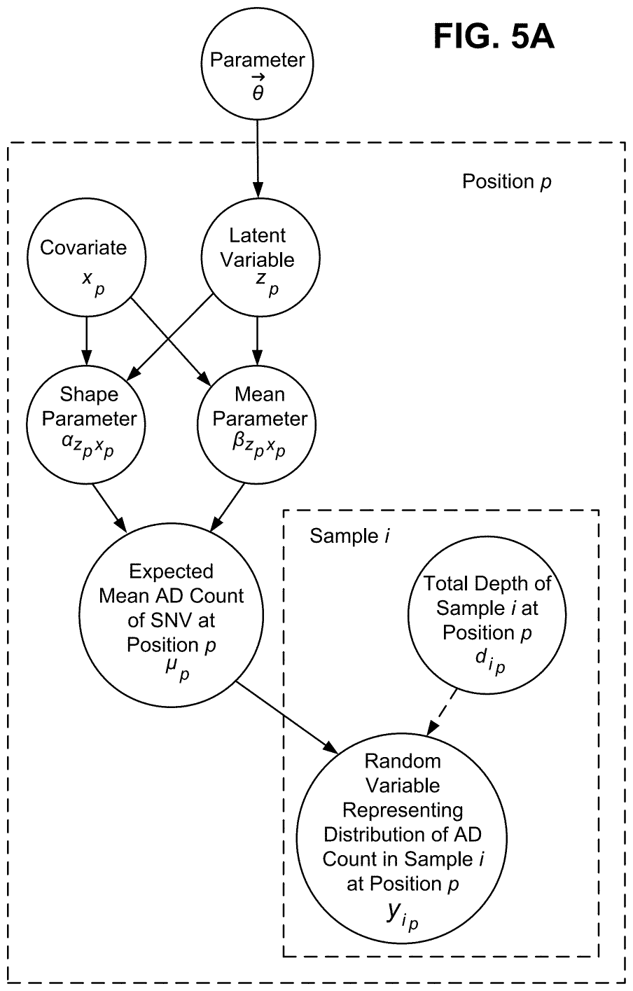

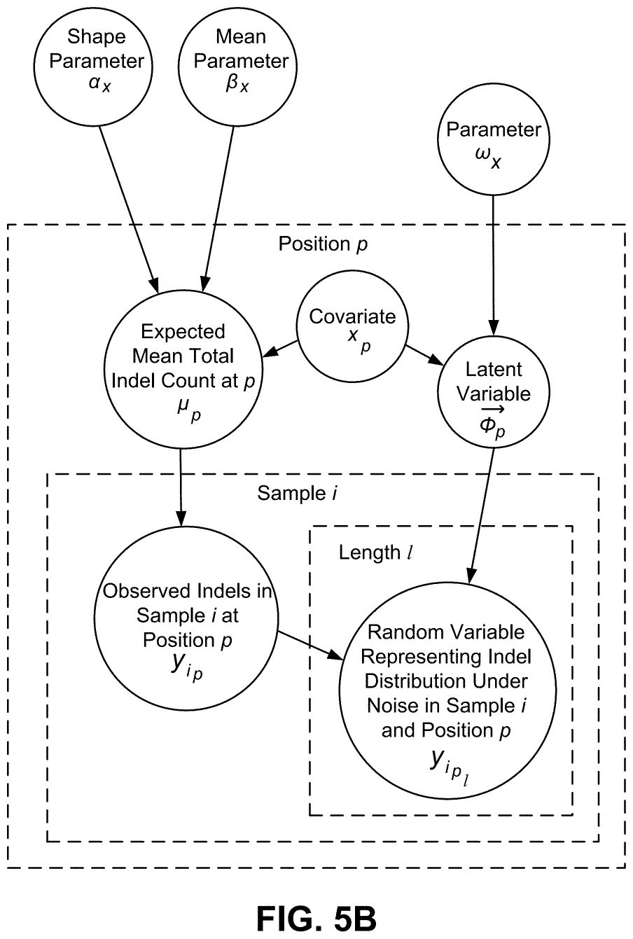

FIG. 5A shows dependencies between parameters and sub-models of a Bayesian hierarchical model 225 for determining true single nucleotide variants according to some embodiments. Parameters of models can be stored in the parameter database 230. In the example shown in FIG. 5A, \vec{θ} represents the vector of weights assigned to each mixture component. The vector \vec{θ} takes on values within the simplex in K dimensions and can be learned or updated via posterior sampling during training. It can be given a uniform prior on said simplex for such training. The mixture component to which a position p belongs can be modeled by latent variable zₚ using one or more different multinomial distributions:

zₚ˜Multinom(\vec{θ})

Together, the latent variable zₚ, the vector of mixture components \vec{θ}, α, and β allow the model for μ, that is, a sub-model of the Bayesian hierarchical model 225, to have parameters that “pool” knowledge about noise, that is they represent similarity in noise characteristics across multiple positions. Thus, positions of sequence reads can be pooled or grouped into latent classes by the model. Also advantageously, samples of any of these “pooled” positions can help train these shared parameters. A benefit of this is that the processing system 200 can determine a model of noise in healthy samples even if there is little to no direct evidence of alternative alleles having been observed for a given position previously (e.g., in the healthy tissue samples used to train the model).

The covariate xₚ (e.g., a predictor) encodes known contextual information regarding position p which can include, but is not limited to, information such as trinucleotide context, mappability, segmental duplication, or other information associated with sequence reads. Trinucleotide context can be based on a reference allele and can be assigned numerical (e.g., integer) representation. For instance, “AAA” is assigned 1, “ACA” is assigned 2, “AGA” is assigned 3, etc. Mappability represents a level of uniqueness of alignment of a read to a particular target region of a genome. For example, mappability is calculated as the inverse of the number of position(s) where the sequence read will uniquely map. Segmental duplications correspond to long nucleic acid sequences (e.g., having a length greater than approximately 1000 base pairs) that are nearly identical (e.g., greater than 90% match) and occur in multiple locations in a genome as result of natural duplication events (e.g., not associated with a cancer or disease).

The expected mean AD count of a SNV at position p is modeled by the parameter μₚ. For sake of clarity in this description, the terms μₚ and yₚ refer to the position specific sub-models of the Bayesian hierarchical model 225. In some embodiments, μₚ is modeled as a Gamma-distributed random variable having shape parameter α_{z}_{ₚ}_{,x}_{ₚ} and mean parameter β_{z}_{ₚ}_{,x}_{ₚ}:

μₚ˜Gamma(α_{z}_{ₚ}_{,x}_{ₚ},β_{z}_{ₚ}_{,x}_{ₚ})

In other embodiments, other functions can be used to represent μₚ, examples of which include but are not limited to: a log-normal distribution with log-mean γ_{z}_{ₚ} and log-standard-deviation σ_{z}_{ₚ}_{,x}_{ₚ}, a Weibull distribution, a power law, an exponentially-modulated power law, or a mixture of the preceding.

In the example shown in FIG. 5A, the shape and mean parameters are each dependent on the covariate xₚ and the latent variable zₚ, though in other embodiments, the dependencies can be different based on various degrees of information pooling during training. For instance, the model can alternately be structured so that α_{z}_{ₚ} depends on the latent variable but not the covariate. The distribution of AD count of the SNV at position p in a human population sample i (of a healthy individual) is modeled by the random variable yᵢ_{ₚ}. In some embodiments, the distribution is a Poisson distribution given a depth dᵢₚ of the sample at the position:

yᵢ_{ₚ}|dᵢₚ˜Poisson(dᵢ_{ₚ}˜μₚ)

In other embodiments, other functions can be used to represent yᵢ_{ₚ}, examples of which include but are not limited to: negative binomial, Conway-Maxwell-Poisson distribution, zeta distribution, and zero-inflated Poisson.

FIG. 5B shows dependencies between parameters and sub-models of a Bayesian hierarchical model for determining true insertions or deletions according to some embodiments. In contrast to the SNV model shown in FIG. 5A, the model for indels shown in FIG. 5B includes different levels of hierarchy. The covariate xₚ encodes known features at position p and can include, e.g., a distance to a homopolymer, distance to a RepeatMasker repeat, or other information associated with previously observed sequence reads. Latent variable \vec{ϕ}ₚ can be modeled by a Dirichlet distribution based on parameters of vector \vec{ω}ₓ, which represent indel length distributions at a position and can be based on the covariate. In some embodiments, \vec{ω}ₓ is also shared across positions (\vec{ω}_{x,p}) that share the same covariate value(s). Thus for example, the latent variable can represent information such as that homopolymer indels occur at positions 1, 2, 3, etc. base pairs from the anchor position, while trinucleotide indels occur at positions 3, 6, 9, etc. from the anchor position.

The expected mean total indel count at position p is modeled by the distribution μₚ. In some embodiments, the distribution is based on the covariate and has a Gamma distribution having shape parameter αₓ_{ₚ} and mean parameter βₓ_{ₚ}_{z}_{ₚ}:

μₚ˜Gamma(αₓ_{ₚ},βₓ_{ₚ}_{,z}_{ₚ})

In other embodiments, other functions can be used to represent μₚ, examples of which include but are not limited to: negative binomial, Conway-Maxwell-Poisson distribution, zeta distribution, and zero-inflated Poisson.

The observed indels at position p in a human population sample i (of a healthy individual) is modeled by the distribution yᵢ_{ₚ}. Similar to example in FIG. 5A, in some embodiments, the distribution of indel intensity is a Poisson distribution given a depth dᵢ_{ₚ} of the sample at the position:

yᵢ_{ₚ}|dᵢ_{ₚ}˜Poisson(dᵢ_{ₚ}·μₚ)

In other embodiments, other functions can be used to represent yᵢ_{ₚ}, examples of which include but are not limited to: negative binomial, Conway-Maxwell-Poisson distribution, zeta distribution, and zero-inflated Poisson.

Due to the fact that indels can be of varying lengths, an additional length parameter is present in the indel model that is not present in the model for SNVs. As a consequence, the example model shown in FIG. 5B has an additional hierarchical level (e.g., another sub-model), which is again not present in the SNV models discussed above. The observed count of indels of length l (e.g., up to 100 or more base pairs of insertion or deletion) at position p in sample i is modeled by the random variable yᵢ_{ₚᵢ}, which represents the indel distribution under noise conditional on parameters. The distribution can be a multinomial given indel intensity yᵢ_{ₚ} of the sample and the distribution of indel lengths \vec{ϕ}ₚ at the position:

\vec{y}ᵢ_{ₚᵢ}|yᵢ_{ₚ},\vec{ϕ}ₚ˜Multinom(yᵢ_{ₚ},\vec{ϕ}ₚ)

In other embodiments, a Dirichlet-Multinomial function or other types of models can be used to represent yᵢ_{ₚᵢ}.

By architecting the model in this manner, the machine learning engine 220 can decouple learning of indel intensity (i.e., noise rate) from learning of indel length distribution. Independently determining inferences for an expectation for whether an indel will occur in healthy samples and expectation for the length of the indel at a position can improve the sensitivity of the model. For example, the length distribution can be more stable relative to the indel intensity at a number of positions or regions in the genome, or vice versa.

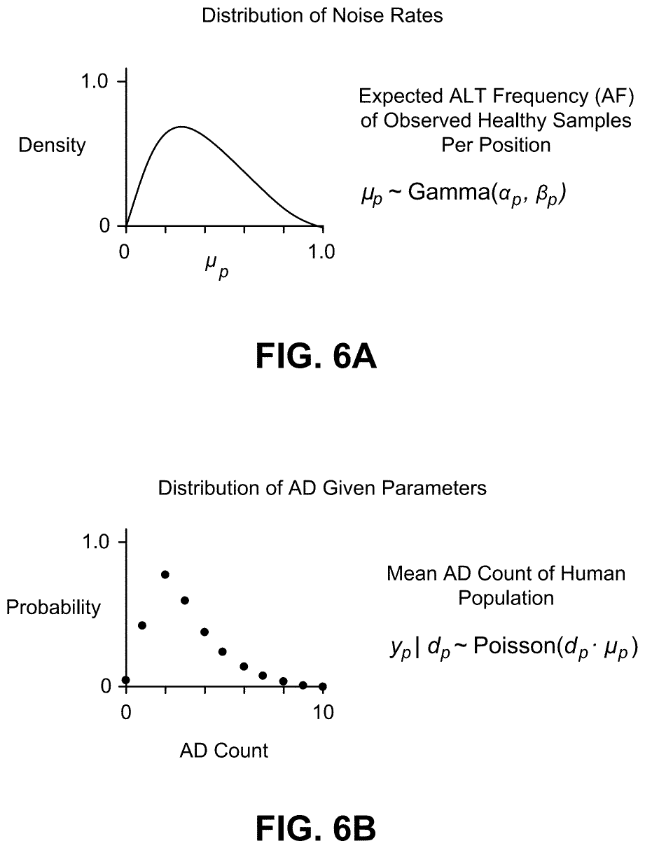

FIG. 6A shows an example diagram of noise rates associated with a Bayesian hierarchical model 225 according to some embodiments. FIG. 6B shows an example diagram of alternate depth (AD) associated with a Bayesian hierarchical model 225 according to some embodiments. The graph shown in FIG. 6A depicts the distribution μₚ of noise rates, i.e., likelihood (or intensity) of SNVs or indels for a given position as characterized by a model. The continuous distribution represents the expected AF μₚ of non-cancer or non-disease mutations (e.g., mutations naturally occurring in healthy tissue) based on training data of observed healthy samples from healthy individuals (e.g., retrieved from the sequence database 210). Though not shown in FIG. 6A, in some embodiments, the shape and mean parameters of μₚ can be based on other variables such as the covariate xₚ or latent variable zₚ. The graph shown in FIG. 6B depicts the distribution of AD at a given position for a sample of a subject, given parameters of the sample such as sequencing depth dₚ at the given position. The discrete probabilities for a draw of μₚ are determined based on the predicted true mean AD count of the human population based on the expected mean distribution μₚ.

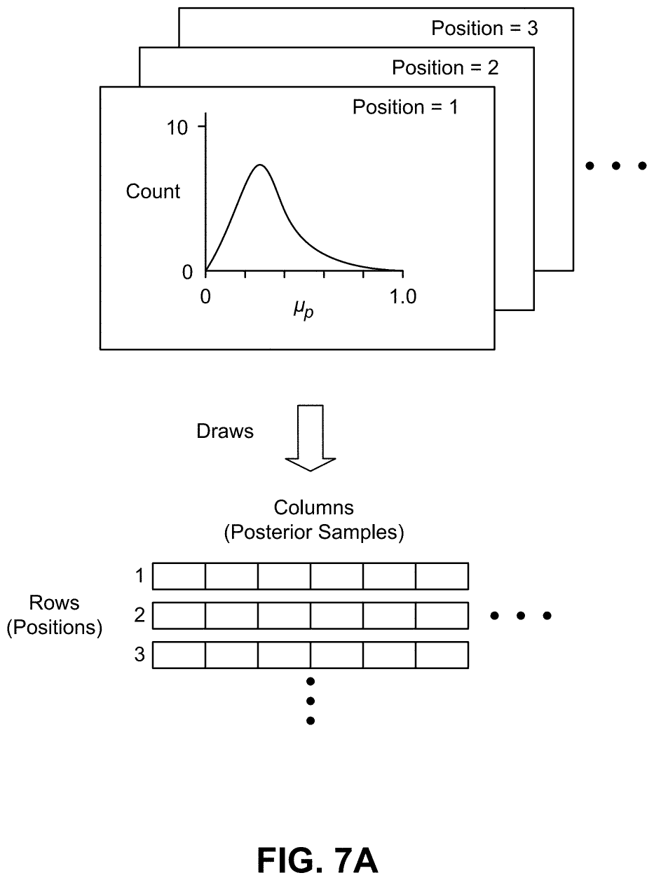

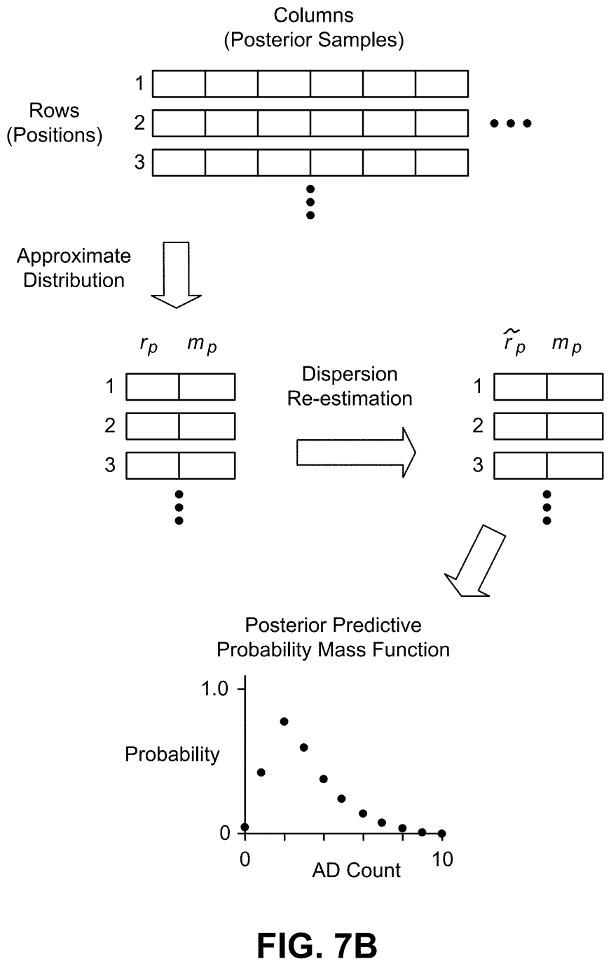

FIG. 7A is a diagram of an example process for determining parameters by fitting a Bayesian hierarchical model 225 according to some embodiments. To train a model, the machine learning engine 220 samples iteratively from a posterior distribution of expected noise rates (e.g., the graph shown in FIG. 6B) for each position of a set of positions. The machine learning engine 220 can use Markov chain Monte Carlo (MCMC) methods for sampling, e.g., a Metropolis-Hastings (MH) algorithm, custom MH algorithms, Gibbs sampling algorithm, Hamiltonian mechanics-based sampling, random sampling, among other sampling algorithms. During Bayesian Inference training, parameters are drawn from the joint posterior distribution to iteratively update all (or some) parameters and latent variables of the model (e.g., \vec{θ}, zₚ, α_{z}_{ₚ}_{,x}_{ₚ}, β_{z}_{ₚ}_{,x}_{ₚ}, μₚ, etc.).

In some embodiments, the machine learning engine 220 performs model fitting by storing draws of μₚ, the expected mean counts of AF per position and per sample, in the parameters database 230. The model is trained or fitted through posterior sampling, as previously described. In some embodiments, the draws of μₚ are stored in a matrix data structure having a row per position of the set of positions sampled and a column per draw from the joint posterior (e.g., of all parameters conditional on the observed data). The number of rows R can be greater than 6 million and the number of columns for N iterations of samples can be in the thousands. In other embodiments, the row and column designations are different than the embodiment shown in FIG. 7A, e.g., each row represents a draw from a posterior sample, and each column represents a sampled position (e.g., transpose of the matrix example shown in FIG. 7A).

FIG. 7B is a diagram of using parameters from a Bayesian hierarchical model 225 to determine a likelihood of a false positive according to some embodiments. The machine learning engine 220 can reduce the R rows-by-N column matrix shown in FIG. 7A into an R rows-by-2 column matrix illustrated in FIG. 7B. In some embodiments, the machine learning engine 220 determines a dispersion parameter rₚ (e.g., shape parameter) and mean parameter mₚ (which can also be referred to as a mean rate parameter mₚ) per position across the posterior samples μₚ. The dispersion parameter rₚ can be determined as

$${r}_{p} = \frac{{m}_{p}^{2}}{{v}_{p}^{2}} ,$$

where mₚ and vₚ are the mean and variance of the sampled values of μₚ at the position, respectively. Those of skill in the art will appreciate that other functions for determining rₚ may also be used such as a maximum likelihood estimate.

The machine learning engine 220 can also perform dispersion re-estimation of the dispersion parameters in the reduced matrix, given the mean parameters. In some embodiments, following Bayesian training and posterior approximation, the machine learning engine 220 performs dispersion re-estimation by retraining for the dispersion parameters [symbol] based on a negative binomial maximum likelihood estimator per position. The mean parameter can remain fixed during retraining. In some embodiments, the machine learning engine 220 determines the dispersion parameters r′ₚ at each position for the original AD counts of the training data (e.g., yᵢ_{ₚ} and dᵢ_{ₚ} based on healthy samples). The machine learning engine 220 determines [symbol] =max(rₚ, r′ₚ), and stores [symbol] in the reduced matrix. Those of skill in the art will appreciate that other functions for determining [symbol] can also be used, such as a method of moments estimator, posterior mean, or posterior mode.

During application of trained models, the processing system 200 can access the dispersion (e.g., shape) parameters [symbol] and mean parameters mₚ to determine a function parameterized by [symbol] and mₚ. The function can be used to determine a posterior predictive probability mass function (or probability density function) for a new sample of a subject. Based on the predicted probability of a certain AD count at a given position, the processing system 200 can account for site-specific noise rates per position of sequence reads when detecting true positives from samples. Referring back to the example use case described with respect to FIG. 4, the PDFs shown for Mutations A and B can be determined using the parameters from the reduced matrix of FIG. 7B. The posterior predictive probability mass functions can be used to determine the probability of samples for Mutations A or B having an AD count at certain position.

### V. Example Process Flows for Noise Models

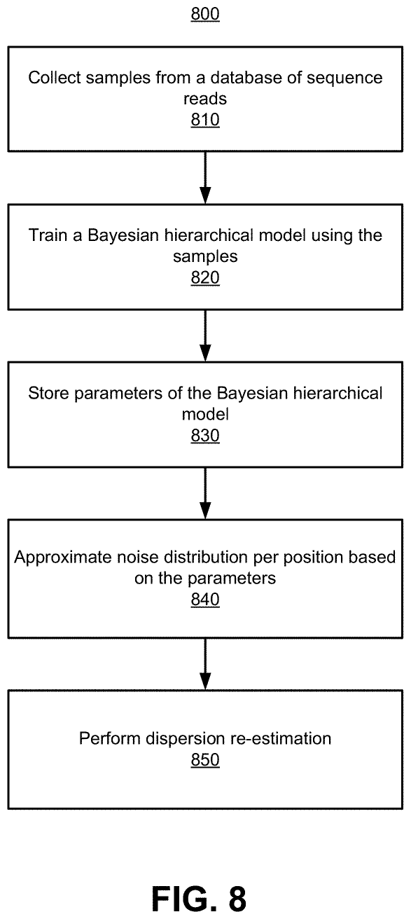

FIG. 8 is flowchart of a method 800 for training a Bayesian hierarchical model 225 according to some embodiments. In step 810, the machine learning engine 220 collects samples, e.g., training data, from a database of sequence reads (e.g., the sequence database 210). In step 820, the machine learning engine 220 trains the Bayesian hierarchical model 225 using the samples using a Markov Chain Monte Carlo method. During training, the model 225 can keep or reject sequence reads conditional on the training data. The machine learning engine 220 can exclude sequence reads of healthy individuals that have less than a threshold depth value or that have an AF greater than a threshold frequency in order to remove suspected germline mutations that are not indicative of target noise in sequence reads. In other embodiments, the machine learning engine 220 can determine which positions are likely to contain germline variants and selectively exclude such positions using thresholds like the above. In some embodiments, the machine learning engine 220 can identify such positions as having a small mean absolute deviation of AFs from germline frequencies (e.g., 0, ½, and 1).

The Bayesian hierarchical model 225 can update parameters simultaneously for multiple (or all) positions included in the model. Additionally, the model 225 can be trained to model expected noise for each ALT. For instance, a model for SNVs can perform a training process four or more times to update parameters (e.g., one-to-one substitutions) for mutations of each of the A, T, C, and G bases to each of the other three bases. In step 830, the machine learning engine 220 stores parameters of the Bayesian hierarchical model 225 (e.g., ensemble parameters output by the Markov Chain Monte Carlo method). In step 840, the machine learning engine 220 approximates noise distribution (e.g., represented by a dispersion parameter and a mean parameter) per position based on the parameters. In step 850, the machine learning engine 220 performs dispersion re-estimation (e.g., maximum likelihood estimation) using original AD counts from the samples (e.g., training data) used to train the Bayesian hierarchical model 225.

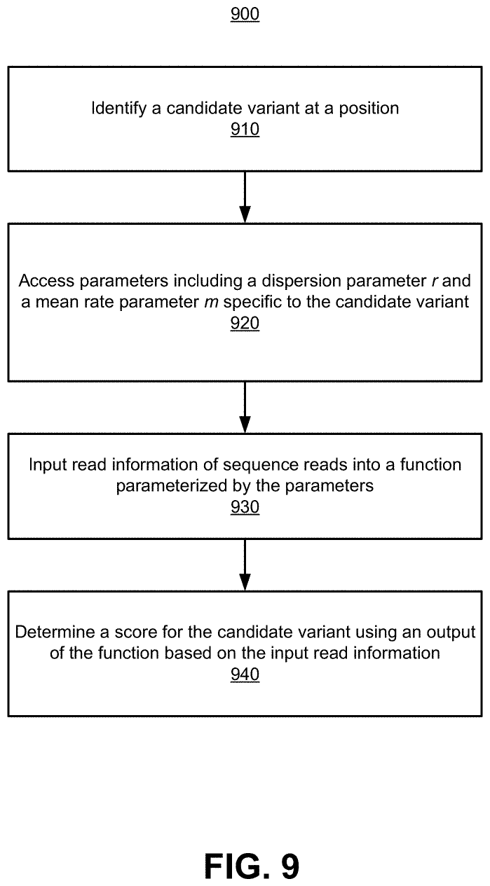

FIG. 9 is flowchart of a method 900 for determining a likelihood of a false positive according to some embodiments. At step 910, the processing system 200 identifies a candidate variant, e.g., at a position p of a sequence read, from a set of sequence reads, which can be sequenced from a cfDNA sample obtained from an individual. At step 920, the processing system 200 accesses parameters, e.g., dispersion and mean rate parameters [symbol] and mₚ, respectively, specific to the candidate variant, which can be based on the position p of the candidate variant. The parameters can be derived using a model, e.g., a Bayesian hierarchical model 225 representing a posterior predictive distribution with an observed depth of a given sequence read and a mean parameter μₚ at the position p as input. In some embodiments, the mean parameter μₚ is a gamma distribution encoding a noise level of nucleotide mutations with respect to the position p for a training sample.

At step 930, the processing system 200 inputs read information (e.g., AD or AF) of the set of sequence reads into a function (e.g., based on a negative binomial) parameterized by the parameters, e.g., [symbol] and mₚ. At step 940, the processing system 200 (e.g., the score engine 235) determines a score for the candidate variant (e.g., at the position p) using an output of the function based on the input read information. The score can indicate a likelihood of seeing an allele count for a given sample (e.g., from a subject) that is greater than or equal to a determined allele count of the candidate variant (e.g., determined by the model and output of the function). The processing system 200 can convert the likelihood into a Phred-scaled score. In some embodiments, the processing system 200 uses the likelihood to determine false positive mutations responsive to determining that the likelihood is less than a threshold value. In some embodiments, the processing system 200 uses the function to determine that a sample of sequence reads includes at least a threshold count of alleles corresponding to a gene found in sequence reads from a tumor biopsy of an individual. Responsive to this determination, the processing system 200 can predict presence of cancer cells in the individual based on variant calls. In some embodiments, the processing system 200 can perform weighting based on quality scores, use the candidate variants and quality scores for false discovery methods, annotate putative calls with quality scores, or provision to subsequent systems.

The processing system 200 can use functions encoding noise levels of nucleotide mutations with respect to a given training sample for downstream analysis. In some embodiments, the processing system 200 uses the aforementioned negative binomial function parameterized by the dispersion and mean rate parameters [symbol] and mₚ to determine expected noise for a particular nucleic acid position within a sample, e.g., cfDNA or gDNA. Moreover, the processing system 200 can derive the parameters by training a Bayesian hierarchical model 225 using training data associated with the particular nucleic acid sample. The embodiments below describe another type of model referred to herein as a joint model 225, which can use output of a Bayesian hierarchical model 225.

### VI. Example Joint Model

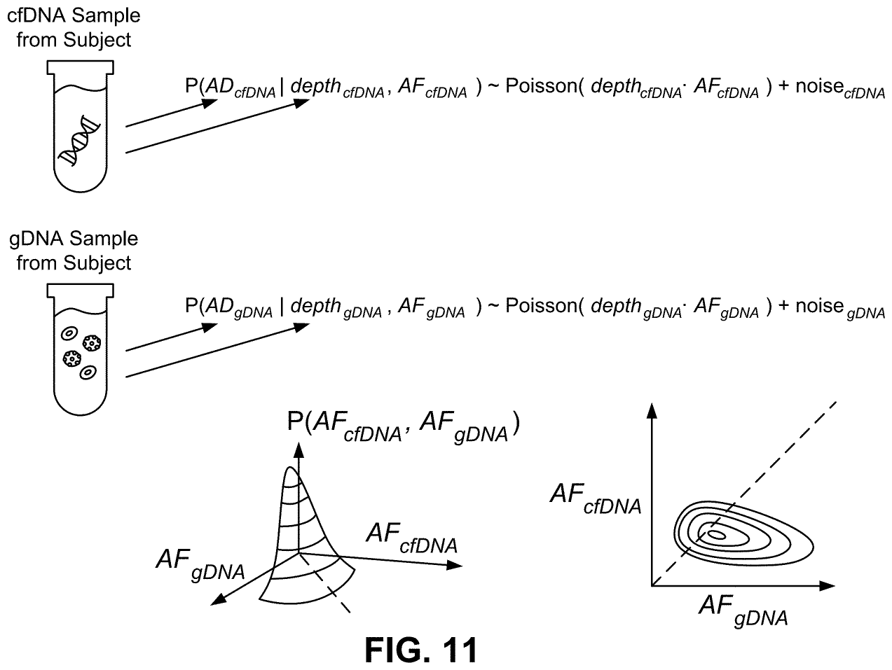

FIG. 10 is flowchart of a method 1000 for using a joint model (e.g., model 225) to process cell free nucleic acid (e.g., cfDNA) samples and genomic nucleic acid (e.g., gDNA) samples according to some embodiments. The joint model 225 can be independent of positions of nucleic acids of cfDNA and gDNA. The method 1000 can be performed in conjunction with the methods 800 and/or 900 shown in FIGS. 8-9. For example, the methods 800 and 900 are performed to determine noise of nucleotide mutations with respect to cfDNA and gDNA samples of training data from health samples. FIG. 11 is a diagram of an application of a joint model according to some embodiments. Steps of the method 1000 are described below with reference to FIG. 11.

In step 1010, the sequence processor 205 determines depths and ADs for the various positions of nucleic acids from the sequence reads obtained from a cfDNA sample of a subject. The cfDNA sample can be collected from a sample of blood plasma from the subject. Step 1010 can include previously described steps of the method 100 shown in FIG. 1.

In step 1020, the sequence processor 205 determines depths and ADs for the various positions of nucleic acids from the sequence reads obtained from a gDNA of the same subject. The gDNA may be collected from white blood cells or a tumor biopsy from the subject. Step 1020 may include previously described steps of the method 100 shown in FIG. 1.

VI. A. Example Signal of Joint Model

In step 1030, a joint model 225 determines a likelihood of a “true” AF of the cfDNA sample of the subject by modeling the observed ADs for cfDNA. In some embodiments, the joint model 225 uses a Poisson distribution function, parameterized by the depths observed from the sequence reads of cfDNA and the true AF of the cfDNA sample, to model the probability of observing a given AD in cfDNA of the subject (also shown in FIG. 11). The product of the depth and the true AF may be the rate parameter of the Poisson distribution function, which represents the mean expected AF of cfDNA.

P(AD_{cfDNA}|depth_{cfDNA},AF_{cfDNA})˜Poisson(depth_{cfDNA}·AF_{cfDNA})+noise_{cfDNA}

The noise component noise_{cfDNA} is further described below in section VI. B. Example Noise of Joint Model. In other embodiments, other functions can be used to represent AD_{cfDNA}, examples of which include but are not limited to: negative binomial, Conway-Maxwell-Poisson distribution, zeta distribution, and zero-inflated Poisson.

In step 1040, the joint model 225 determines a likelihood of a “true” AF of the gDNA sample of the subject by modeling the observed ADs for gDNA. In some embodiments, the joint model 225 uses a Poisson distribution function parameterized by the depths observed from the sequence reads of gDNA and the true AF of the gDNA sample to model the probability of observing a given AD in gDNA of the subject (also shown in FIG. 11). The joint model 225 can use a same function for modeling the likelihoods of true AF of gDNA and cfDNA, though the parameter values differ based on the values observed from the corresponding sample of the subject.

P(AD_{gDNA}|depth_{gDNA},AF_{gDNA})˜Poisson(depth_{gDNA}·AF_{gDNA})+noise_{gDNA}

The noise component noise_{gDNA} is further described below in section VI. B. Example Noise of Joint Model. In other embodiments, other functions may be used to represent AD_{gDNA}, examples of which include but are not limited to: negative binomial, Conway-Maxwell-Poisson distribution, zeta distribution, and zero-inflated Poisson.

Since the true AF of cfDNA, as well as the true AF of gDNA, are inherent properties of the biology of a particular subject, it may not necessarily be practical to determine an exact value of the true AF from either source. Moreover, various sources of noise also introduce uncertainty into the estimated values of the true AF. Accordingly, the joint model 225 uses numerical approximation to determine the posterior distributions of true AF conditional on the observed data (e.g., depths and ADs) from a subject and corresponding noise parameters:

P(AF_{cfDNA}|depth_{cfDNA},AD_{cfDNA}, [symbol] _{cfDNA},mₚ_{_{cfDNA}})

P(AF_{gDNA}|depth_{gDNA},AD_{gDNA}, [symbol] _{gDNA},mₚ_{_{gDNA}})

The joint model 225 determines the posterior distributions using Bayes' theorem with a prior, for example, a uniform distribution. The priors used for cfDNA and gDNA can be the same (e.g., a uniform distribution ranging from 0 to 1) and independent from each other.

In some embodiments, the joint model 225 determines the posterior distribution of true AF of cfDNA using a likelihood function by varying the parameter, true AF of cfDNA, given a fixed set of observed data from the sample of cfDNA. Additionally, the joint model 225 determines the posterior distribution of true AF of gDNA using another likelihood function by varying the parameter, true AF of gDNA, given a fixed set of observed data from the sample of gDNA. For both cfDNA and gDNA, the joint model 225 numerically approximates the output posterior distribution by fitting a negative binomial (NB):

$$P ( AF \vert depth , AD ) ∝ {\sum }_{i = 0}^{AD} \frac{{{e}^{- AF \cdot depth} ( AF \cdot depth )}^{i}}{i !} \cdot NB ( AD - i , size = r , μ = m \cdot depth )$$

In some embodiments, the joint model 225 performs numerical approximation using the following parameters for the negative binomial, which can provide an improvement in computational speed:

P(AF|depth,AD)∝NB(AD,size=r,μ=m·depth)

Where:

m=AF+m

r=r·m²/m²

Since the observed data is different between cfDNA and gDNA, the parameters determined for the negative binomial of cfDNA will vary from those determined for the negative binomial of gDNA.

In step 1050, the variant caller 240 determines, using the likelihoods, a probability that the true AF of the cfDNA sample is greater than a function of the true AF of the gDNA sample. The function can include one or more parameters, for example, empirically-determined k and p values stored in the parameter database 230. The probability represents a confidence level that at least some nucleotide mutations from the sequence reads of cfDNA are not found in sequence reads of reference tissue. The variant caller 240 can provide this information to other processes for downstream analysis. For instance, a high probability indicates that nucleotide mutations from a subject's sequence reads of cfDNA and that are not found in sequence reads of gDNA may have originated from a tumor or other source of cancer within the subject. In contrast, low probability indicates that nucleotide mutations observed in cfDNA likely did not originate from potential cancer cells or other diseased cells of the subject. Instead, the nucleotide mutations can be attributed to naturally occurring mutations in healthy individuals, due to factors such as germline mutations, clonal hematopoiesis (unique mutations that form subpopulations of blood cell DNA), mosaicism, chemotherapy or mutagenic treatments, technical artifacts, among others.

In some embodiments, the variant caller 240 determines that the posterior probability satisfies a chosen criteria based on the one or more parameters (e.g., k and p described below). The distributions of variants are conditionally independent given the sequences of the cfDNA and gDNA. That is, the variant caller 240 presumes that ALTs and noise present in one of the cfDNA or gDNA sample is not influenced by those of the other sample, and vice versa. Thus, the variant caller 240 considers the probabilities of the expected distributions of AD as independent events in determining the probability of observing both a certain true AF of cfDNA and a certain true AF of gDNA, given the observed data and noise parameters from both sources:

P(AF_{cfDNA},AF_{gDNA}|depth,AD, [symbol] ,mₚ)∝

P(AF_{cfDNA}|depth_{cfDNA},AD_{cfDNA}, [symbol] _{cfDNA},mₚ_{_{cfDNA}})·

P(AF_{gDNA}|depth_{gDNA},AD_{gDNA}, [symbol] _{gDNA},mₚ_{_{gDNA}})

In the example 3D plot in FIG. 11, the probability P(AF_{cfDNA}, AF_{gDNA}) is plotted as a 3D contour for pairs of AF_{cfDNA} and AF_{gDNA} values. The example 2D slice of the 3D contour plot along the AF_{cfDNA} and AF_{gDNA} axis illustrates that the volume of the contour plot is skewed toward greater values of AF_{gDNA} relative to the values of AF_{cfDNA}. In other embodiments, the contour plot can be skewed differently or have a different form than the example shown in FIG. 11. To numerically approximate the joint likelihood, the sequence processor 205 can calculate the volume defined by the 3D contour of P(AF_{cfDNA}, AF_{gDNA}) and a boundary line illustrated by the dotted line shown in the plots of FIG. 11. The sequence processor 205 determines the slope of the boundary line according to the k parameter value, and the boundary line intersects the origin point. The k parameter value can account for a margin of error in the determined true AF. Particularly, the margin of error can cover naturally occurring mutations in healthy individuals such as germline mutations, clonal hematopoiesis, loss of heterozygosity, and other sources as described above. Since the 3D contour is split by the boundary line, at least a portion of variants detected from the cfDNA sample can potentially be attributed to variants detected from the gDNA sample, while another portion of the variants can potentially be attributed to a tumor or other source of cancer.

In some embodiments, the sequence processor 205 determines that a given criteria is satisfied by the posterior probability by determining the portion of the joint likelihood that satisfies the given criteria. The given criteria can be based on the k and p parameter, where p represents a threshold probability for comparison. For example, the sequence processor 205 determines the posterior probability that true AF of cfDNA is greater than or equal to the true AF of gDNA multiplied by k, and whether the posterior probability is greater than p:

$$P ( {AF}_{cfDNA} \geq k \cdot {AF}_{gDNA} ) > p , where$$

$$P ( {AF}_{cfDNA} \geq k \cdot {AF}_{gDNA} ) = {\int }_{0}^{1} {\int }_{k \cdot {AF}_{gDNA}}^{1} {f}_{cfDNA} ( {AF}_{cfDNA} ) \cdot {f}_{gDNA} ( {AF}_{gDNA} ) d {AF}_{cfDNA} d {AF}_{gDNA} = {\int }_{0}^{1} {f}_{gDNA} ( {AF}_{gDNA} ) [ {\int }_{k \cdot {AF}_{gDNA}}^{1} {f}_{cfDNA} ( {AF}_{cfDNA} ) \cdot d {AF}_{cfDNA} ] {dAF}_{gDNA} = {\int }_{0}^{1} {f}_{gDNA} ( {AF}_{gDNA} ) ( 1 - {F}_{cfDNA} ( k \cdot {AF}_{gDNA} ) ) {dAF}_{gDNA}$$

As shown in the above equations, the sequence processor 205 determines a cumulative sum F_{cfDNA} of the likelihood of the true AF of cfDNA. Furthermore, the sequence processor 205 integrates over the likelihood function of the true AF of gDNA. In another embodiment, the sequence processor 205 determines the cumulative sum for the likelihood of the true AF of gDNA, and integrates over the likelihood function of the true AF of cfDNA. By calculating the cumulative sum of one of the two likelihoods (e.g., building a cumulative distribution function), instead of computing a double integral over both likelihoods for cfDNA and gDNA, the sequence processor 205 reduces the computational resources (expressed in terms of compute time or other similar metrics) required to determine whether the joint likelihood satisfies the criteria and can also increase precision of the calculation of the posterior probability.

VI. B. Example Noise of Joint Model

To account for noise in the estimated values of the true AF introduced by noise in the cfDNA and gDNA samples, the joint model 225 can use other models of the processing system 200 previously described with respect to FIGS. 4-9. In some embodiments, the noise components shown in the above equations for P(AD_{cfDNA}|depth_{cfDNA}, AF_{cfDNA}) and P(AD_{gDNA}|depth_{gDNA}, AF_{gDNA}) are determined using Bayesian hierarchical models 225, which can be specific to a candidate variant (e.g., SNV or indel). Moreover, the Bayesian hierarchical models 225 can cover candidate variants over a range of specific positions of nucleotide mutations or indel lengths.

In some examples, the joint model 225 uses a function parameterized by cfDNA-specific parameters to determine a noise level for the true AF of cfDNA. The cfDNA-specific parameters can be derived using a Bayesian hierarchical model 225 trained with a set of cfDNA samples, e.g., from healthy individuals. In addition, the joint model 225 uses another function parameterized by gDNA-specific parameters to determine a noise level for the true AF of gDNA. The gDNA-specific parameters can be derived using another Bayesian hierarchical model 225 trained with a set of gDNA samples, e.g., from the same healthy individuals. In some embodiments, the functions are negative binomial functions having a mean parameter m and dispersion parameter f, and can also depend on the observed depths of sequence reads from the training samples:

noise_{cfDNA}=NB(m_{cfDNA}·depth_{cfDNA},\tilde{r}_{cfDNA})

noise_{gDNA}=NB(m_{gDNA}·depth_{gDNA},\tilde{r}_{gDNA})

In other embodiments, the sequence processor 225 can use a different type of function and types of parameters for cfDNA and/or gDNA. Since the cfDNA-specific parameters and gDNA-specific parameters are derived using different sets of training data, the parameters can be different from each other and particular to the respective type of nucleic acid sample. For instance, cfDNA samples can have greater variation in AF than gDNA samples, and thus \tilde{r}_{cfDNA} can be greater than \tilde{r}_{gDNA}.

VI. C. Example Parameters for Joint Model

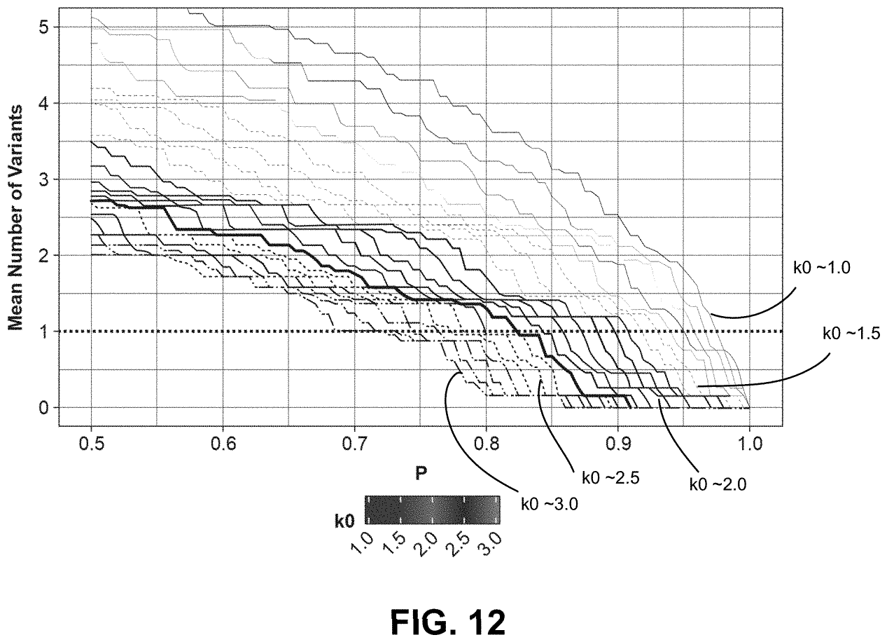

FIG. 12 is a diagram of observed counts of variants in samples from healthy individuals according to some embodiments. Each data point corresponds to a position (across a range of nucleic acid positions) of a given one of the individuals. The parameters k and p used by the joint model 225 for joint likelihood computations can be selected empirically (e.g., to tune sensitivity thresholds) by cross-validating with sets of cfDNA and gDNA samples from healthy individuals and/or samples known to have cancer. The example results shown in FIG. 12 were obtained with Assay B and using blood plasma samples for the cfDNA and white blood cell samples for the gDNA. For given parameter values for k (“k0” as shown in FIG. 12) and p, the diagram plots a mean number of variants, which represents a computed upper confidence bound (UCB) of false positives for the corresponding sample. The diagram indicates that the number of false positives decreases as the value of p increases. In addition, the plotted curves have greater numbers of false positives for lower values of k, e.g., closer to 1.0. The dotted line indicates a target of one variant, though the empirical results show that the mean number of false positives mostly fall within the range of 1-5 variants, fork values between 1.0 and 5.0, and p values between 0.5 and 1.0.

The selection of parameters can involve a tradeoff between a target sensitivity (e.g., adjusted using k and p) and target error (e.g., the upper confidence bound). For given pairs of k and p values, the corresponding mean number of false positives can be similar in value, though the sensitivity values can exhibit greater variance. In some embodiments, the sensitivity is measured using percent positive agreement (PPA) values for tumor, in contrast to PPA for cfDNA, which can be used as a measurement of specificity:

$${PPA}_{tumor} = \frac{tumor + cfDNA}{tumor}$$

$${PPA}_{cfDNA} = \frac{tumor + cfDNA}{cfDNA}$$

In the above equations, “tumor” represents the number of mean variant calls from a ctDNA sample using a set of parameters, and “cfDNA” represents the number of mean variant calls from the corresponding cfDNA sample using the same set of parameters.

In some embodiments, cross-validation is performed to estimate the expected fit of the joint model 225 to sequence reads (for a given type of tissue) that are different from the sequence reads used to train the joint model 225. For example, the sequence reads can be obtained from tissues having lung, prostate, and breast cancer, etc. To avoid or reduce the extent of overfitting the joint model 225 for any given type of cancer tissue, parameter values derived using samples of a set of types of cancer tissue are used to assess statistical results of other samples known to have a different type of cancer tissue. For instance, parameter values for lung and prostate cancer tissue are applied to a sample having breast cancer tissue. In some embodiments, one or more lowest k values from the lung and prostate cancer tissue data that maximizes the sensitivity is selected to be applied to the breast cancer sample. Parameter values can also be selected using other constraints such as a threshold deviation from a target mean number of false positives, or 95% UCB of at most 3 per sample. The processing system 200 can cycle through multiple types of tissue to cross validate sets of cancer-specific parameters.

### VII. Example Mixture Model

VII. A. Example Process Flows

Other types of models other than the joint model discussed above can be used to determine whether a candidate variant is a novel somatic mutation, or a mutation arising from another source, such as from noise or from blood-matched genomic samples. One such model is referred to herein as a “mixture model.” The mixture model determines predictions of sources of candidate variants by using the properties present in populations of variants to determine whether a candidate variant in question has properties that are more similar to those of novel somatic mutations, or those of other sources such as variants matched in genomic DNA samples. The machine learning engine 220 can use any number of training data sets to train a mixture model (e.g., model 225).

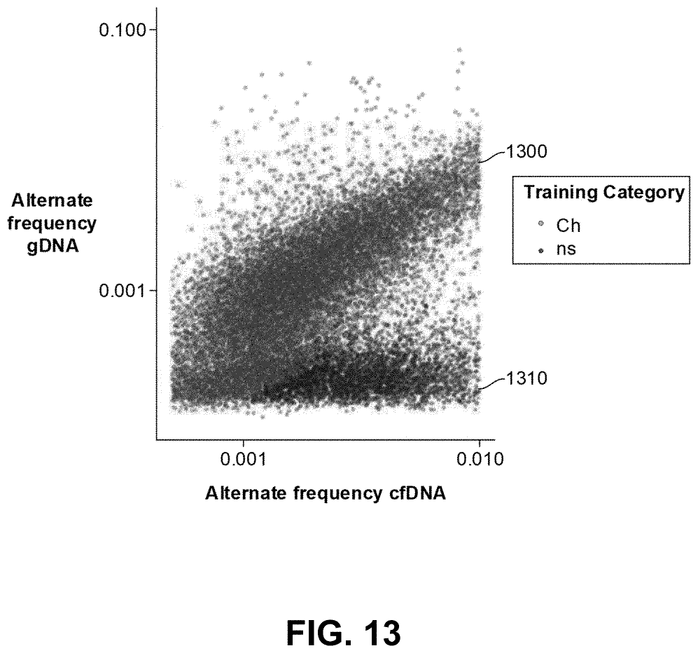

FIG. 13 illustrates an example of why such a model can be useful. In the example shown in FIG. 13, the first distribution 1300 includes variants attributed to clonal hematopoiesis, which can be matched to somatic mutations observed in white blood cells. In other embodiments, other types of mutations associated with sources different than clonal hematopoiesis can also be used in a training data set. For instance, somatic mutations can be attributed to variants detected in sequence reads from tissue samples of skin cells or other types of cells in a human body. The second distribution 1310 includes variants attributed to novel somatic mutations.

FIG. 13 illustrates distributions of training data sets having different types of mutations according to one embodiment. As illustrated, the first distribution 1310 and the second distribution 1320 have different properties such as shape and variance when plotted on with respect to AF cfDNA and AF gDNA axes. FIG. 13 illustrates that AF does provide some meaningful information for distinguishing variants as the distributions mostly do not overlap, however the shape and variance of the distributions indicate that AF is not the only property of the data that can be indicative of the source of a candidate variant. The mixture model is designed to incorporate these other properties.

The machine learning engine 220 trains a mixture model 225 to determine classifications of candidate variants, where the classifications represent predictions of a source of a given candidate variant. Though the example shown in FIG. 13 includes two possible sources (e.g., clonal hematopoiesis or novel somatic variant) of a candidate variant, in other embodiments, a model 225 can be trained using different training sets or more than two training sets, where each training set can correspond to a different source for classification. The embodiment of FIG. 13 includes axes representing alternate frequency in gDNA and cfDNA for illustrative purposes. In practice, a mixture model 225 can analyze any number of properties of distributions of variants or other metadata describing the variants, not limited to alternate frequency.

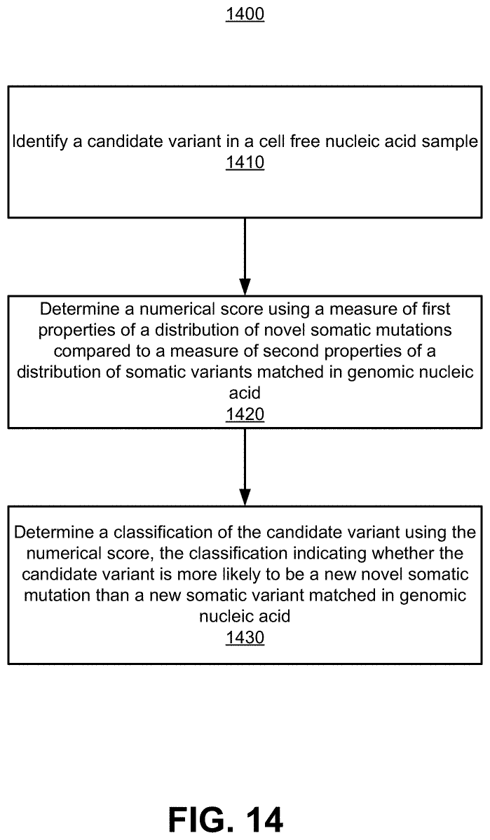

FIG. 14 is flowchart of a method 1400 for classifying candidate variants in nucleic acid samples according to some embodiments. At step 1410, the processing system 100 identifies a candidate variant in a cell free nucleic acid sample. At step 1420, a trained mixture model 225 determines a numerical score using a measure of first properties of a distribution of novel somatic mutations compared to a measure of second properties of a distribution of somatic variants matched in genomic nucleic acid. For example, the somatic variants can be matched with white blood cells in the case of clonal hematopoiesis, or matched with tumor-derived variants detected from a tissue sample. The properties can include depth, alternate frequency, or trinucleotide context of sequence reads of a sample used to determine the corresponding distribution. In embodiments including more than two possible sources of the candidate variant, the numerical score can be determined by comparing the first and second properties to any number of additional properties of a distribution of variants associated with the other possible sources.

The properties of the distributions can be modeled by generalized linear models (GLMs) using a gamma distribution. Additionally, the mixture model 225 can determine the numerical score by modeling allele counts of the candidate variant using a Poisson distribution after a gamma distribution. The measure based on comparison of the properties can represent a likelihood under a generalized linear model using a gamma distribution with Poisson counts. In some embodiments, the numerical score can be adjusted by modifying the likelihood under the generalized linear model by an empirical adjustment factor. Empirical adjustment is further described below in Section VII. C. Example Tuning of Mixture Model.

At step 1430, the mixture model 225 determines a classification of the candidate variant using the numerical score. The classification indicates whether the candidate variant is more likely to be a new novel somatic mutation than a new somatic variant matched in genomic nucleic acid. In some embodiments, the mixture model 225 classifies a candidate variant as a novel somatic variant responsive to determining that the numerical score is greater than a threshold score. For instance, the numerical score represents a probability that the candidate variant is a novel somatic variant, and the threshold score is 40%, 50%, 60%, etc. In some embodiments, the mixture model 225 determines a numerical score for each potential source, e.g., 55% novel somatic variant and 45% clonal hematopoiesis. In some embodiments, the probabilities of being attributed to the possible sources sum to 100%, though not necessarily. Moreover, if numerical scores are less than or equal to the threshold score, the mixture model 225 can determine to not classify a candidate variant (or classify as having an unknown or inconclusive source) due to the candidate variant not resembling variants from either the novel somatic variant or clonal hematopoiesis sources.

In some embodiments, the processing system 100 determines a prediction that the candidate variant is a true mutation in the cell free nucleic acid sample based on the classification. Additionally, the processing system 100 can determine a likelihood that an individual has a disease based on in part on the prediction. The nucleic acid sample can be obtained from the individual, and the nucleic acid sample can be processed using any number of the previously described assay steps, e.g., labeling fragments with UMIs, performing enrichment, or generating sequence reads. The disease can be associated with a particular type of cancer or health condition. In some embodiments, the method 1400 includes determining a diagnosis or treatment based on the likelihood. Furthermore, the method 1400 can also include performing a treatment on the individual to remediate the disease.

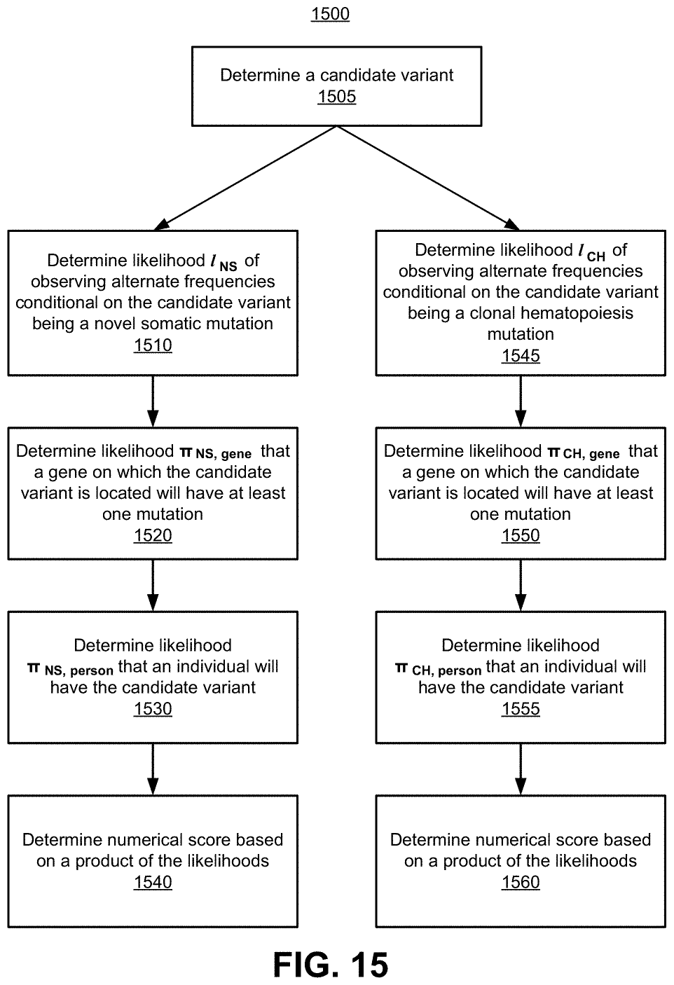

FIG. 15 is flowchart of a method 1500 for determining numerical scores for candidate variants according to some embodiments. The method 1500 can be used in conjunction with the method 1400 of FIG. 14. In particular, steps of the method 1500 can be used to determine the numerical score in step 1420 of the method 1400. In step 1505, a candidate variant of an individual is determined.

In step 1510, a mixture model 225 determines an observational likelihood l_{NS} of observing alternate frequencies conditional on the candidate variant being a novel somatic mutation. In other words, given the observed alternate frequency in cfDNA and gDNA (e.g., white blood cells), the mixture model 225 determines the observational likelihood l_{NS} that a given candidate is the novel somatic mutation unmatched in white blood cell, or another type of genomic nucleic acid sample. The observational likelihood l_{NS} can be determined based on data observed in a sample population (e.g., an intended use population).

In step 1520, the mixture model 225 determines a gene-specific likelihood π_{NS,gene}, i.e., a likelihood that a gene on which the candidate variant is located will have at least one mutation. The gene-specific likelihood indicates a relative likelihood that a mutation falls within a gene given (e.g., conditional on) a particular mutation process or type (e.g., novel somatic or clonal hematopoiesis), which can be estimated based on data from a sample population. Accounting for gene-specific likelihoods can improve accuracy of the mixture model 225 because mutations arising from different processes can be more or less likely to occur in specific genes. For example, mutations arising from clonal hematopoiesis can be more likely to occur within DNMT3A than in other genes. Additionally, the TP53 gene can have a greater observed number of mutations relative to other genes.

In step 1530, the mixture model 225 determines a person-specific likelihood π_{NS,person} that an individual will have the candidate variant, given that the likelihoods in steps 1510 and 1520 are held equal, e.g., conditional on a ratio of novel somatic mutations to clonal hematopoiesis mutations within the individual. The person-specific likelihood is determined per individual, while the likelihoods in steps 1510 and 1520 are per population. The person-specific likelihood indicates an expected rate of a mutation (e.g., novel somatic π_{NS,person} or clonal hematopoiesis π_{CH,person}) within the individual, coming from a mutational process (e.g., novel somatic or clonal hematopoiesis). For example, 90% of the observed mutations within the individual are derived from clonal hematopoiesis.

Steps 1510-1530 can be repeated to determine likelihoods of observing a clonal hematopoiesis mutation. For example, in step 1545, the mixture model 225 determines an observational likelihood l_{CH} of observing alternate frequencies conditional on the candidate variants being a clonal hematopoiesis mutation, e.g., estimated using data observed in a sample population. In step 1550, the mixture model 225 determines a gene-specific likelihood π_{CH,gene}, that a gene on which the candidate variant is located will have at least one clonal hematopoiesis mutation. In step 1555, the mixture model 225 determines a person-specific likelihood π_{CH,person} that an individual will have the candidate variant, given that the clonal hematopoiesis-based likelihoods in steps 1545 and 1550 are held equal.

In steps 1540 and 1560, the mixture model 225 determines the numerical scores 1″ for novel somatic and clonal hematopoiesis mutations based on a product of the above corresponding likelihoods, i.e., from steps 1510-1530 and steps 1545-1555, respectively:

l″_{NS}=l_{NS}(alt_{gDNA},alt_{cfDNA})×π_{NS,gene}×π_{NS,person}

l″_{CH}=l_{CH}(alt_{gDNA},alt_{cfDNA})×π_{CH,gene}×π_{CH,person}

Determination by the mixture model 225 of the likelihoods of observed variants in cfDNA and gDNA, gene-specific likelihoods, and person-specific likelihoods is further detailed below in Section VII. B. Example Mixture Model Calculations.

VII. B. Example Mixture Model Calculations

Referring back to the example distributions shown in FIG. 13, a mixture model 225 of the processing system 100 can be trained to determine likelihoods of observing variants in individual cfDNA and gDNA samples while distinguishing by source of the variants. In some embodiments where two possible sources are novel somatic (NS) mutations and clonal hematopoiesis (CH), the mixture model 225 determines the following likelihoods l′_{NS} and l′_{CH} under the two distributions using corresponding gene-specific likelihoods and observed likelihoods based on alternate frequency (alt_{gDNA}, alt_{cfDNA}):

l′_{CH}=π_{CH,gene}×l_{CH}(alt_{gDNA},alt_{cfDNA})

l′_{NS}=π_{CH,gene}×l_{NS}(alt_{gDNA},alt_{cfDNA})

The person-specific likelihoods of observing the candidate variant in the gene are represented by π_{CH,person} and π_{NS,person}. The person-specific likelihoods are unknown parameters calculated using an expectation-maximization (EM) algorithm with l′_{CH} and l′_{NS} as input. The number of reads having a certain alternate allele, or alternate allele count, in each of gDNA and cfDNA is represented by alt_{gDNA} and alt_{cfDNA}, respectively. Assuming that the likelihoods sum to one, in some embodiments, the mixture model 225 can determine the numerical score (which may also be referred to as a “responsibility”) for a particular variant as:

$$numerical {score}_{CH} = {l}_{CH}^{″} = \frac{{π}_{CH , person} \times {l}_{CH}^{′}}{{π}_{CH , person} \times {l}_{CH}^{′} + ( 1 - {π}_{CH , person} ) \times {l}_{NS}^{″}}$$

The distribution of variant (e.g., alternate allele) counts can be modeled based on the previously described Bayesian hierarchical models 225. Particularly, the probability that a candidate variant corresponds to noise in sequence reads can be modeled by a negative binomial distribution having parameters r and m. Additionally, the distribution of signal indicating true positive mutations may be modeled by a Poisson distribution, given the depth and alternate frequency λ:

P(alt_{cfDNA}|depth_{cfDNA},λ_{cfDNA})˜NB(r_{cfDNA},m_{cfDNA})+Poisson(λ_{cfDNA}·depth_{cfDNA})

P(alt_{gDNA}|depth_{gDNA},λ_{gDNA})˜NB(r_{gDNA},m_{gDNA})+Poisson(λ_{gDNA}·depth_{gDNA})

To account for dependency between the alternate frequencies of cfDNA and gDNA, the distribution of the alternate frequency of gDNA is modeled as a gamma random variable Γ, having shape parameter α and rate parameter β, dependent on the alternate frequency of cfDNA:

$${λ}_{gDNA} \sim Γ ( α , β ( {λ}_{cfDNA} ) ) , where$$

$$β = \frac{α}{{β}_{0} + {β}_{1} \cdot {λ}_{cfDNA}}$$

The intercept and slope derived from linear regression of the gamma distribution are represented by β₀ and β₁, respectively.

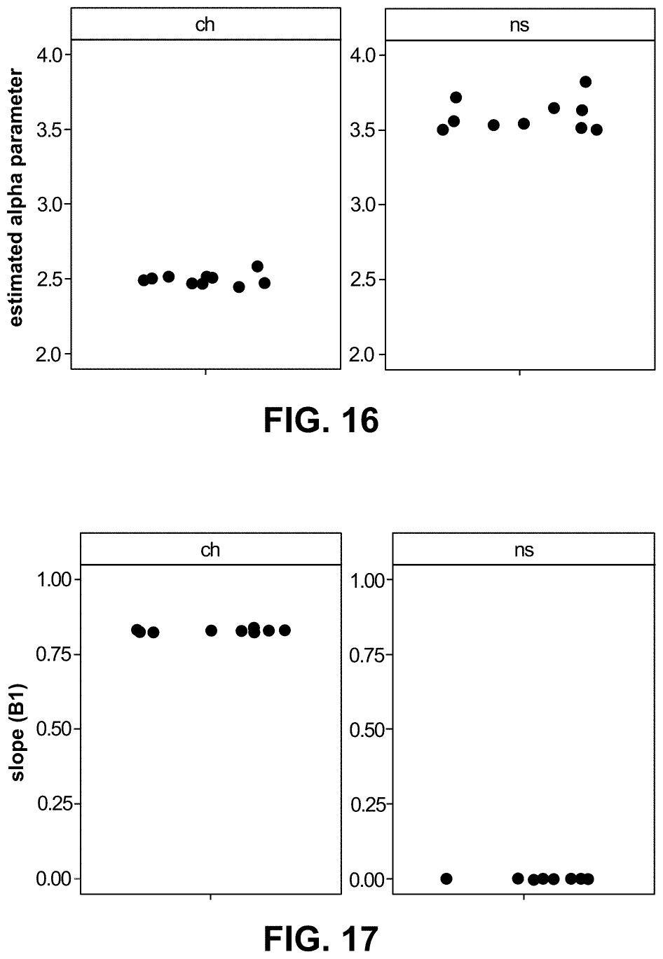

FIG. 16 is a diagram of shape parameters a determined by modeling training data sets having different types of mutations according to some embodiments. FIG. 17 is a diagram of slope parameters β₁ determined by modeling training data sets having different types of mutations according to some embodiments. Each data point represents a cross validation fold from the training data. In the examples shown in FIGS. 16-17, a gamma generalized linear model is used with training data sets labeled as novel somatic or clonal hematopoiesis types of mutations to estimate the model parameters. Distributions for the novel somatic and clonal hematopoiesis training data sets each have a shape parameter α and rate parameter β. The shape parameters a and slopes β₁ are stable as indicated by the low variance in example values shown in FIGS. 16-17. Furthermore, as shown in FIG. 16, novel somatic mutations have greater values for the shape parameters a than the parameter values estimated for clonal hematopoiesis associated mutations (e.g., somatic variants matched with white blood cell gDNA), which can reflect a greater level of variance in properties of the novel somatic mutations relative to properties of the clonal hematopoiesis associated mutations. The greater shape parameter values for novel somatic mutations can also be due to heightened magnitude of ratio-changes near zero. As shown in FIG. 17, clonal hematopoiesis associated mutations have greater values for slope β₁ than the values for novel somatic mutations. The slope β₁ for clonal hematopoiesis is approximately 0.8 because variants in white blood cell can be shed into cfDNA and remain at about 80% of blood at steady state in the processed sample.

The processing system 100 can use the gamma distribution parameters derived using training data sets to determine the distribution of likelihood of observed counts of variants. By integrating over alternate frequency of gDNA λ_{gDNA}, the distribution in terms of alternate frequency of cfDNA can be determined as shown in the following steps. Since the noise distribution is not dependent on λ_{gDNA}, the integral is applied to the signal (e.g., Poisson) term and not the negative binomial term. The gamma distribution F is a conjugate prior for the Poisson distribution, so the integral simplifies to a negative binomial having parameters r_{g} and p_{g}. The probability of λ_{gDNA} is gamma distributed conditional on λ_{cfDNA}: Γ(λ_{gDNA}|λ_{cfDNA}). The negative binomial distribution can be parameterized as NB(r, p) or NB(r, m), where

$$p = \frac{r}{r + m \times d} and$$

$$d = {depth}_{gDNA} \cdot P ( {alt}_{gDNA} \vert {depth}_{dDNA} , {λ}_{cfDNA} ) \sim \int NB ( {r}_{gDNA} , {m}_{gDNA} ) + Poisson ( {λ}_{gDNA} \cdot {depth}_{gDNA} ) Γ ( {λ}_{gDNA} \vert {λ}_{cfDNA} ) d {λ}_{gDNA}$$

$$P ( {alt}_{gDNA} \vert {depth}_{gDNA} , {λ}_{cfDNA} ) \sim NB ( {r}_{gDNA} , {m}_{gDNA} ) + \int Poisson ( {λ}_{gDNA} \cdot {depth}_{gDNA} ) Γ ( {λ}_{gDNA} \vert {λ}_{cfDNA} ) d {λ}_{gDNA}$$

$$P ( {alt}_{gDNA} \vert {depth}_{gDNA} , {λ}_{cfDNA} ) \sim NB ( {r}_{gDNA} , {m}_{gDNA} ) + NB ( {r}_{g} , {p}_{g} ) , where$$

$${r}_{g} = α$$

$${p}_{g} = \frac{\frac{α}{{β}_{0} + {β}_{1} \cdot {λ}_{cfDNA}}}{\frac{α}{{β}_{0} + {β}_{1} \cdot {λ}_{cfDNA}} + {depth}_{gDNA}}$$

The processing system 100 determines the joint likelihood of observed counts of cfDNA and gDNA variants (also referred to herein as a raw likelihood) by integrating the product of the distributions of variant counts for gDNA and cfDNA over values for alternate frequency of cfDNA. The distribution of alternate frequency of gDNA can be substituted with P(alt_{gDNA}|depth_{gDNA}, λ_{cfDNA}) determined above in terms of alternate frequency of cfDNA:

$$l ( {alt}_{gDNA} , {alt}_{cfDNA} ) = \int P ( {alt}_{cfDNA} \vert {depth}_{cfDNA} , {λ}_{cfDNA} ) \times P ( {alt}_{gDNA} \vert {depth}_{gDNA} , {λ}_{cfDNA} ) d {λ}_{cfDNA} = \int [ NB ( {r}_{cfDNA} , {p}_{cfDNA} ) + Poisson ( {λ}_{cfDNA} \cdot {depth}_{cfDNA} ) ] \times [ NB ( {r}_{gDNA} , {p}_{gDNA} ) + NB ( {r}_{g} , {p}_{g} ) ] d {λ}_{cfDNA}$$

The negative binomial and Poisson terms can be simplified using moment matching to yield the following integral. The processing system 100 can calculate the integral numerically, for example, using a trapezoidal sum with step size

$$\frac{1}{{depth}_{cfDNA}} . \\ l ( {alt}_{gDNA} , {alt}_{cfDNA} ) = \int NB ( x , y ) \times NB ( z , w ) d {λ}_{cfDNA} , where$$

$$y = \frac{{r}_{cfDNA} \cdot {p}_{cfDNA} - {r}_{cfDNA} \cdot {p}_{cfDNA}^{2} - {p}_{cfDNA}^{2} \cdot {λ}_{cfDNA} \cdot {depth}_{cfDNA} }{{r}_{cfDNA} - {r}_{cfDNA} \cdot {p}_{cfDNA} - {p}_{cfDNA}^{2} \cdot {λ}_{cfDNA} \cdot {depth}_{cfDNA}}$$

$$x = \frac{y}{1 - y} \times ( \frac{{r}_{cfDNA} - {r}_{cfDNA} \cdot {p}_{cfDNA}}{{p}_{cfDNA}} ) + {λ}_{cfDNA} \cdot {depth}_{cfDNA}$$

$$w = \frac{\frac{{r}_{gDNA} ( 1 - {p}_{gDNA} )}{{p}_{gDNA}} + \frac{{r}_{g} ( 1 - {p}_{g} )}{{p}_{g}}}{\frac{{r}_{gDNA} ( 1 - {p}_{gDNA} )}{{p}_{gDNA}^{2}} + \frac{{r}_{g} ( 1 - {p}_{g} )}{{p}_{g}^{2}}}$$

$$z = \frac{w}{1 - w} \times [ \frac{{r}_{gDNA} ( 1 - {p}_{gDNA} )}{{p}_{gDNA}} + \frac{{r}_{g} ( 1 - {p}_{g} )}{{p}_{g}} ]$$

The output of the integral can represent a likelihood of observing a candidate variant based on alternate frequency of the candidate variant in cfDNA and gDNA, e.g., a raw likelihood. In some embodiments, other sample properties such as trinucleotide context can be used to stratify variants for fitting the gamma generalized linear model. The steps described above can be used in step 1510 of the method 1500 of FIG. 15. At step 1520 of the method 1500, the processing system 100 can determine the gene-specific likelihood of a candidate variant in a given gene i for clonal hematopoiesis (CH) associated mutations and novel somatic (NS) mutations as follows, where rate represents a smoothing rate. The smoothing rate across genes is a multinomial or a Dirichlet prior for a multinomial used to determine a pseudo-count of variants:

$${π}_{CH , gene} = \frac{\# of CH variants in gene i + rate}{total \# of CH variants + {\sum }_{genes} rate}$$

$${π}_{NS , gene} = \frac{rate}{total \# of CH variants + {\sum }_{genes} rate}$$

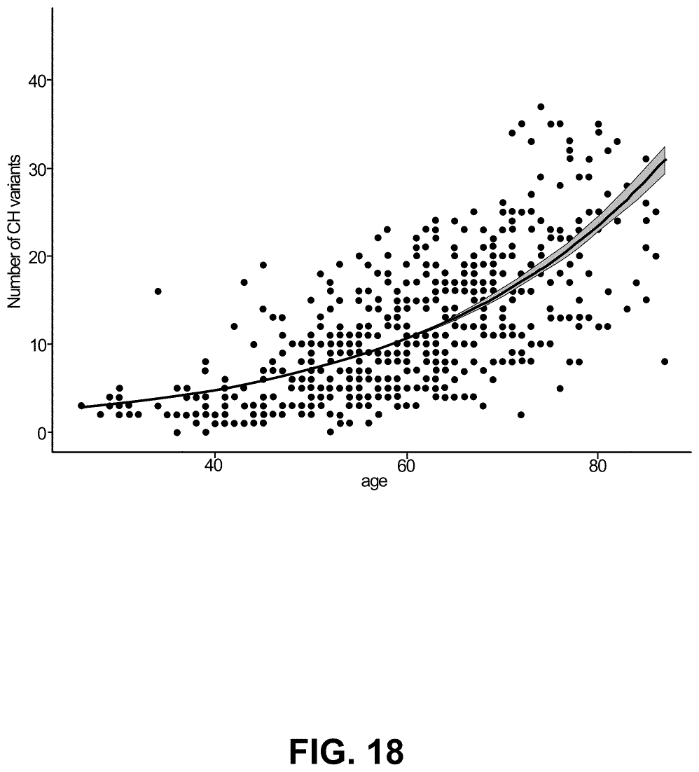

As previously described in step 1530 of the method 1500, the processing system 100 can determine a patient-specific likelihood of having a given candidate variant. In some embodiments, the patient-specific likelihood is initialized to a predetermined value (e.g., 0.5) and updated until convergence during expectation-maximization (EM) iteration. FIG. 18 is a diagram of counts of variants based on age of individuals according to some embodiments. To determine the clonal hematopoiesis variants shown in the example of FIG. 18, the processing system 100 classifies candidate variants as clonal hematopoiesis responsive to determining that the likelihood of observing a clonal hematopoiesis variant is greater than a threshold likelihood, for example, 0.5. The diagram shows that the number of clonal hematopoiesis variants tends to increase with age due to, for instance, somatic mutations that occur naturally over time in individuals.

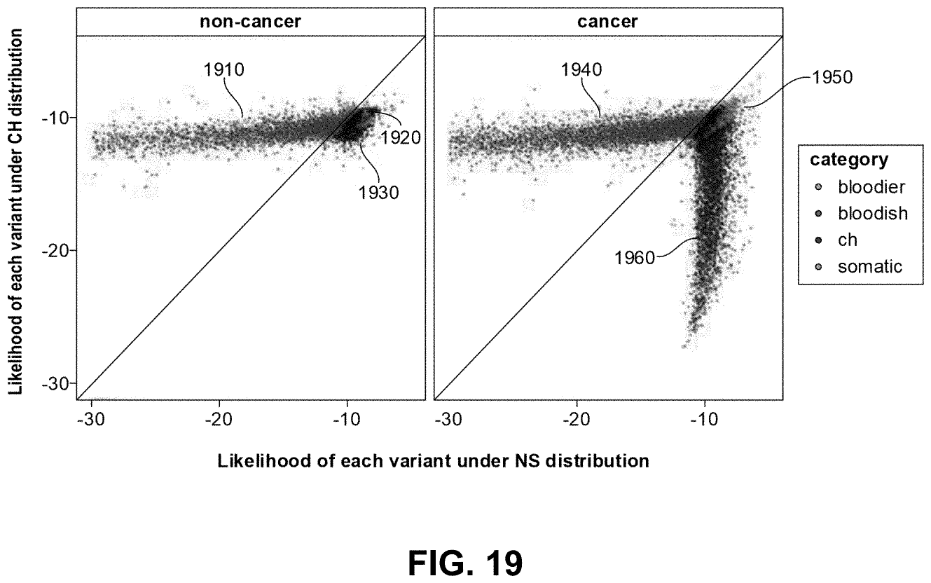

FIG. 19 is a diagram of labeled training data sets according to some embodiments. In some embodiments, the processing system 100 classifies variants into one or more of novel somatic, clonal hematopoiesis, “bloodier,” and “bloodish” categories based on the training data sets. Each data point shown in FIG. 19 represents classification of variants from an individual in a sample. Clonal hematopoiesis variants have evidence of a match with variants observed in white blood cells or other gDNA. The “bloodier” category can include variants that are likely matched with white blood cells, for instance, corresponding to calculated likelihoods greater than a first threshold likelihood. The “bloodish” category can include variants that are possibly matched with white blood cells but less certain than the “bloodier” category, for instance, corresponding to calculated likelihoods greater than a second threshold likelihood that is less than the first threshold likelihood of the “bloodier” category. In the example shown in FIG. 19, the results for non-cancer samples includes a cluster 1910 of clonal hematopoiesis variants, a cluster 1920 of “bloodish” variants, and a cluster 1930 of novel somatic variants. Additionally, the results for cancer samples includes a cluster 1940 of clonal hematopoiesis variants, a cluster 1950 of “bloodish” variants, and a cluster 1960 of novel somatic variants.

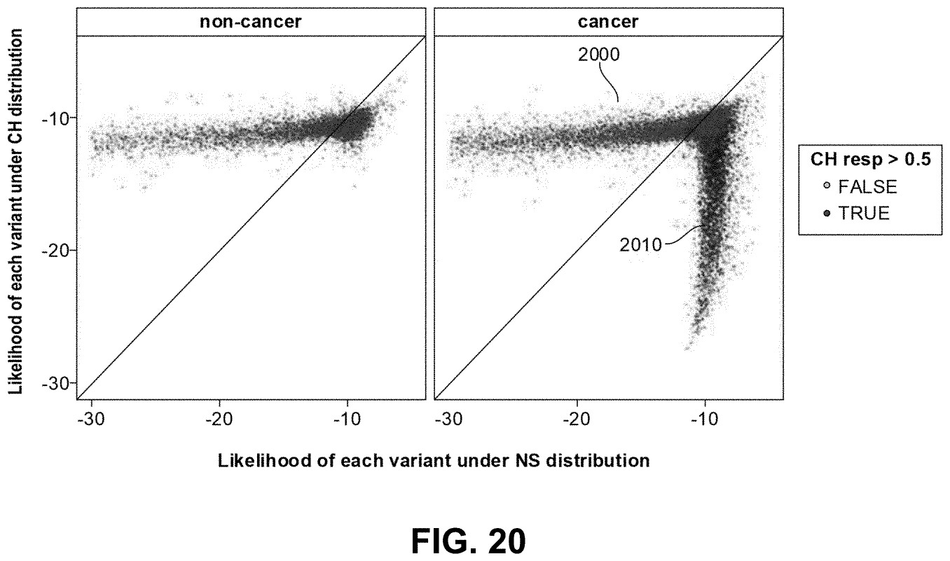

FIG. 20 is another diagram of labeled training data sets according to some embodiments. In contrast to the diagram of FIG. 19 indicating different categories, the diagram of FIG. 20 indicates variants classified based on a threshold likelihood. Particularly, the cluster 2000 of clonal hematopoiesis variants have likelihoods greater than 0.5, the cluster 2010 of novel somatic variants have likelihoods less than or equal to 0.5.

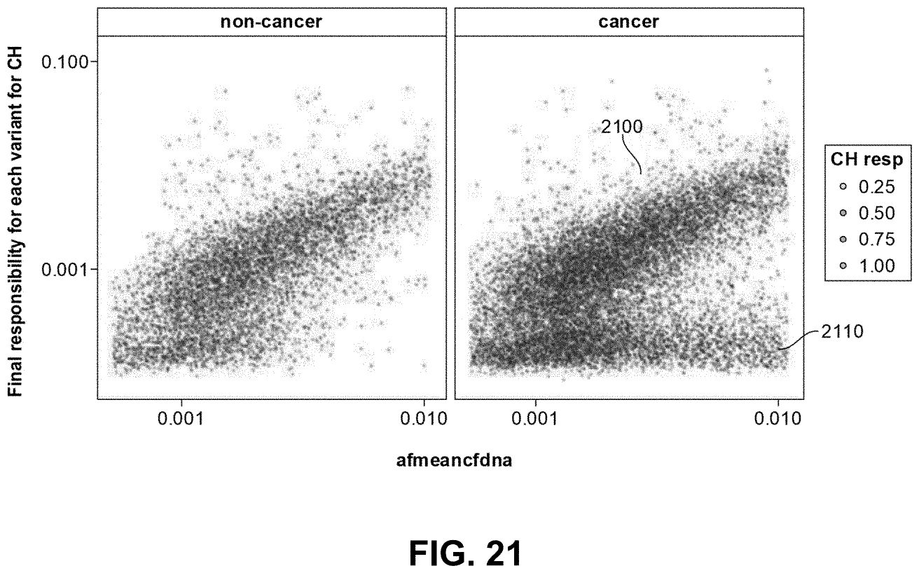

FIG. 21 is a diagram of classified training data sets according to some embodiments. The example diagram shown in FIG. 21 indicates final responsibilities or numerical scores for clonal hematopoiesis variants, which can account for likelihoods based on alternate frequency, gene-specific factors, and person-specific factors. The results for cancer samples includes a cluster 2100 of variants having a 100% likelihood of being clonal hematopoiesis variants and another cluster 2110 of variants with a 25% likelihood of being clonal hematopoiesis variants.

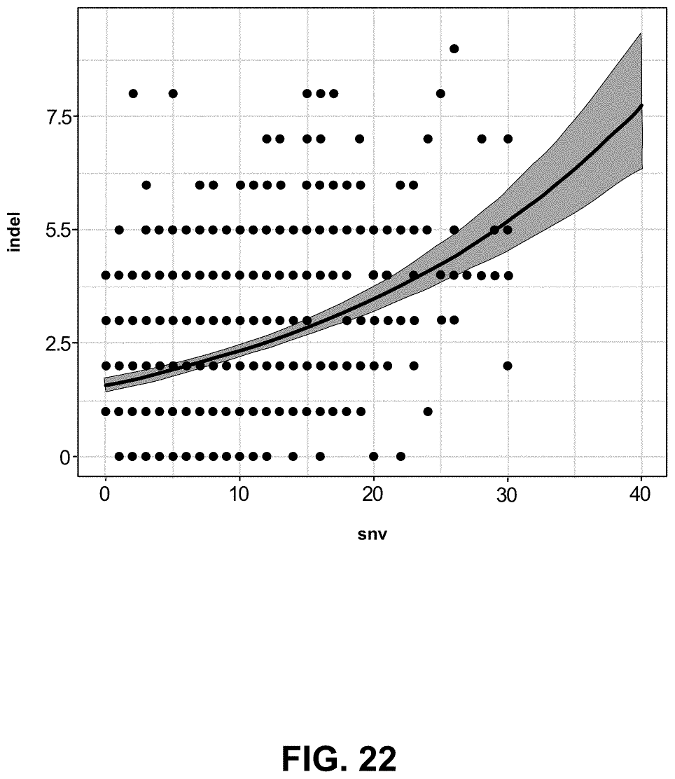

FIG. 22 is a diagram of counts of single nucleotide variants and insertions or deletions sets according to some embodiments. The example results shown in FIG. 22 are determined using training data including samples known to have cancer as well as healthy samples. The processing system 100 matches the variants between gDNA and cfDNA using the processes described above. By comparing a count of observed indels with another count of observed SNVs (e.g., using a threshold clonal hematopoiesis likelihood of 0.5), the processing system 100 can determine that rates of indels and SNVs detected in the training data are correlated at least to some degree, e.g., based on linear regression.

VII. C. Example Tuning of Mixture Model

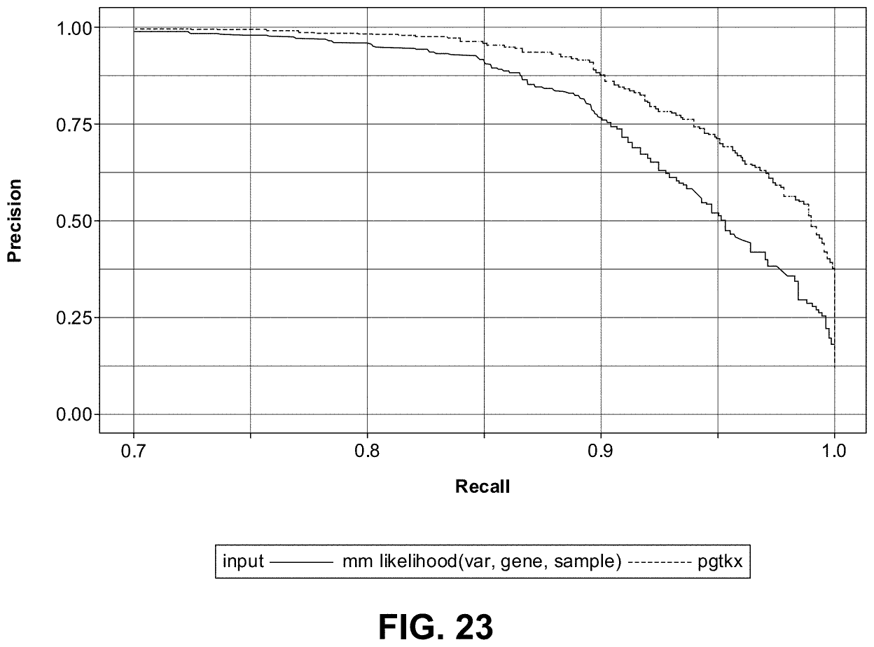

FIG. 23 illustrates a precision recall curve of a model according to some embodiments. The processing system 100 can assess performance of a trained mixture model 225 and tune the mixture model 225 to improve the performance, for instance, by determining an empirical adjustment factor. In some embodiments to assess performance, the processing system 100 generates a precision recall curve (PRC). The precision is a ratio of detected true positives to candidate variants classified as true positive, which can include false positive classifications. The recall (also known as sensitivity) is a ratio of detected true positives to a total number of true positives in a processed sample, which can include false negative classifications missed by the model. The true positives can be determined using tissue biopsy matched variants, and the true negatives can be determined using novel somatic calls in plasma samples from healthy individuals.

The example curves shown in FIG. 23 compare performance of a mixture model 225 to a joint model using a hinge function, denoted by “pgtkx.” The hinge function can be a piecewise function used as a threshold to classify candidate variants as true positives or noise (e.g., false positive). In contrast to the mixture model 225, the hinge function does not compare properties of variants or different sources of potential variants (e.g., clonal hematopoiesis and novel somatic). Since the mixture model 225 may not have been tuned, the performance of the mixture model 225 shown in FIG. 23 falls behind performance of the hinge function. The processing system 100 can improve performance of the mixture model 225 by adjusting the raw likelihood l(alt_{gDNA}, alt_{cfDNA}) using updated parameter values for the gamma generalized linear model, for instance, intercept β₀ and slope β₁. By performing logistic regression with the raw likelihoods as features to predict true positive or true negative calls used in a precision recall curve, the processing system 100 can mitigate sub-optimal parameter values caused by poor calibration.

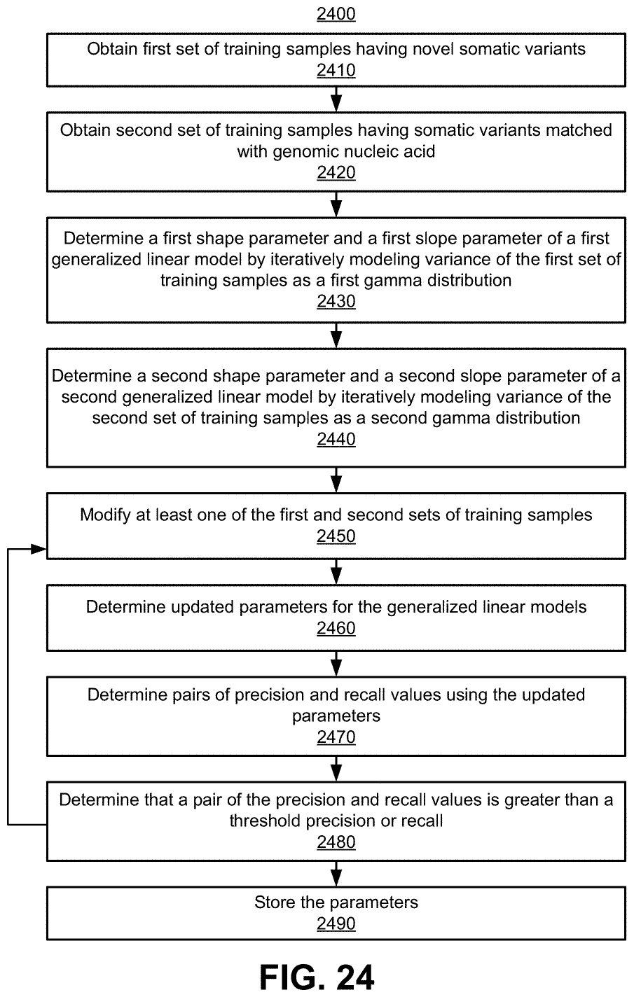

FIG. 24 is flowchart of a method 2400 for tuning a mixture model 225 according to some embodiments. At step 2410, a first set of training samples of novel somatic mutations is obtained. At step 2420, a second set of training samples of somatic variants matched in genomic nucleic acid (e.g., white blood cells) is obtained. At step 2430, the processing system 100 determines a first shape parameter and a first slope parameter of a first generalized linear model by iteratively modeling variance of the first set of training samples as a first gamma distribution. At step 2440, the processing system 100 determines a second shape parameter and a second slope parameter of a second generalized linear model by iteratively modeling variance of the second set of training samples as a second gamma distribution.

In some embodiments, iteratively modeling the variance of the first and second sets of training samples includes step 2450 for modifying the first or second set of training samples using samples of individuals known to have a certain disease (or cancer) or not. At step 2460, the processing system 100 can determine updated parameters for the first and second generalized linear models using one of the modified training samples. At step 2470, the processing system 100 can determine pairs of precision and recall values for predicting true mutations of a test set of cell free nucleic acid samples using the updated parameters. To assess performance of a tuned mixture model 225, at step 2480, the processing system 100 can determine that a precision value is greater than a threshold precision and determine that a recall value (e.g., in a same pair with the precision value) is greater than a threshold recall. The threshold precision and threshold recall can be values from a precision recall curve based on a hinge function, e.g., as shown in FIG. 23. The steps 2450-2480 can be repeated any number of times during empirical tuning of the mixture model 225, for instance, until a certain criteria is satisfied such as the determination in steps 2480 using threshold precision or recall.

At step 2490, the processing system 100 stores the first and second shape parameters and the first and second slope parameters. The stored parameters can be retrieved for use with a mixture model 225 in determining whether a candidate variant is more likely to be a novel somatic mutation than a somatic variant matched in genomic nucleic acid.

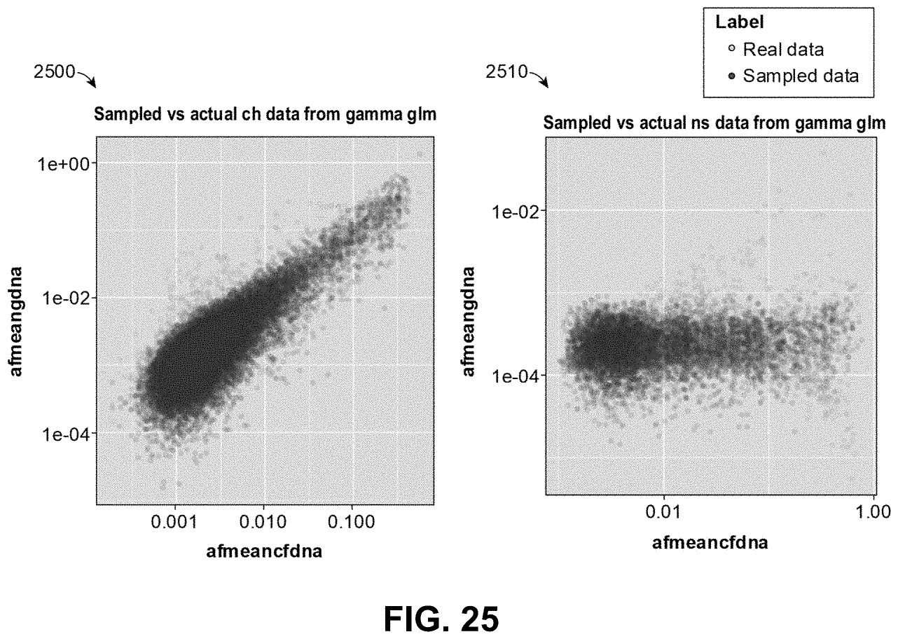

In some embodiments, a mixture model 225 can be empirically tuned using synthetic data. FIG. 25 illustrates distributions of variance for clonal hematopoiesis and novel somatic variants according to one embodiment. The diagram 2500 illustrates clonal hematopoiesis variants called based on a gamma generalized linear model using real data (e.g., from samples obtained from individuals) and sampled (e.g., synthetic) data. The diagram 2510 illustrates novel somatic variants also called based on a gamma generalized linear model using real data and sampled data. In both diagrams, the distributions of called variants based on sampled data have greater variance than the distribution based on real data. A Bayesian hierarchical model may be used to estimate uncertainty of parameters of the gamma generalized linear model. The Bayesian hierarchical model, which accounts for sampling noise, may be: parameterized as:

λ_{gDNA}˜Γ(α,β(λ_{cfDNA}))

alt_{gDNA}|depth_{gDNA}˜Poisson(λ_{d,gDNA}·depth_{gDNA})

alt_{cfDNA}|depth_{cfDNA}˜Poisson(λ_{d,cfDNA}·depth_{cfDNA})

The Poisson distribution is conditional on a particular instance of the true underlying alternate frequency, which depends on a particular unknown λ. The generic distribution can be fitted by drawing λ_{d,gDNA} and λ_{d,cfDNA} across variants and depths.

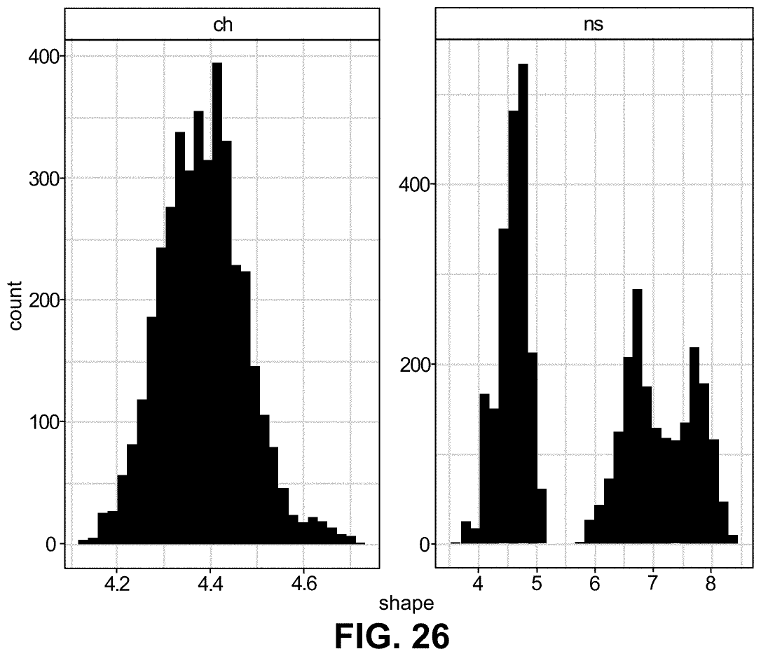

FIG. 26 illustrates histograms of draws from posterior distributions for parameters of distributions of variant calls according to some embodiments. Particularly, the example histograms plot counts of different values of the shape parameter for a gamma distribution used by a mixture model 225 based on sequence reads of a processed sample. The histograms indicate that the novel somatic distribution has greater variance than the clonal hematopoiesis distribution. Furthermore, minor adjustments to bounds of the intercept β₀ or slope β₁ parameters can result in greater changes to the shape parameter estimations for novel somatic variants. These observations can indicate that the gamma generalized linear model does not produce a stable fit of the sample and may be improved by empirical tuning.

In an embodiment, during a process of empirical tuning, a value for the shape parameter α is selected based on precision recall curves from current performance of a mixture model 225. The selected a value is used to determine the person-specific likelihoods for observing a candidate variant, π_{CH}(gene) and π_{NS}(gene). The processing system 100 determines the likelihoods l_{CH} and l_{NS} using a product of the person-specific likelihoods and the raw likelihoods. The likelihoods l_{CH} and l_{NS} can be used to empirically tune a θ parameter. In some embodiments, the observed likelihoods l_{CH} and l_{NS} can be multiplied by the θ parameter to improve discrimination of candidate variants during classification.

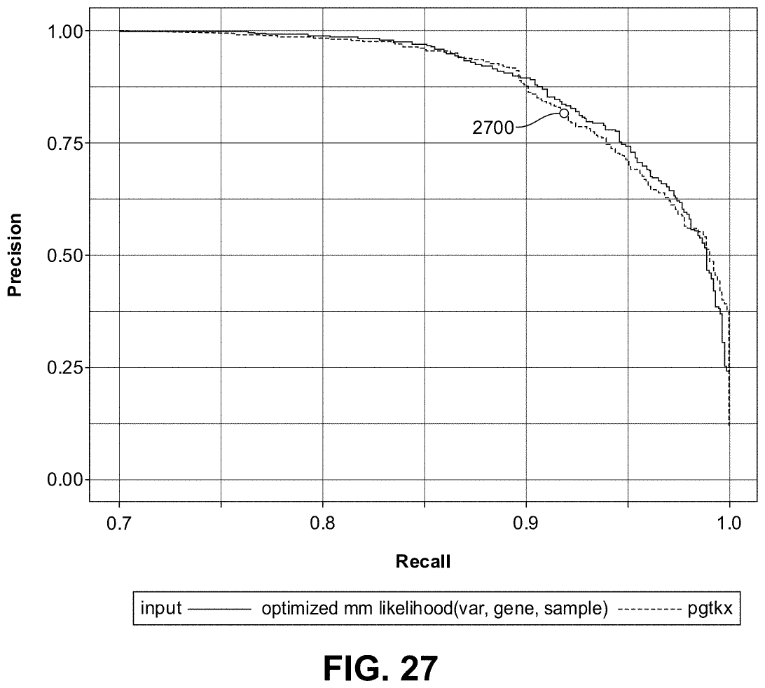

FIG. 27 illustrates another precision recall curve of a tuned model 225 according to some embodiments. The mixture model 225 is empirically tuned with a shape parameter α=50 and θ=5. At a given joint calling threshold point 2700, the tuned mixture model 225 demonstrates improved performance in comparison to the hinge function, i.e., the tuned mixture model 225 has greater values for both precision and recall at point 2700. The mixture model 225 can be tuned for classification rather than estimating values of the parameters or maximum value of training data.

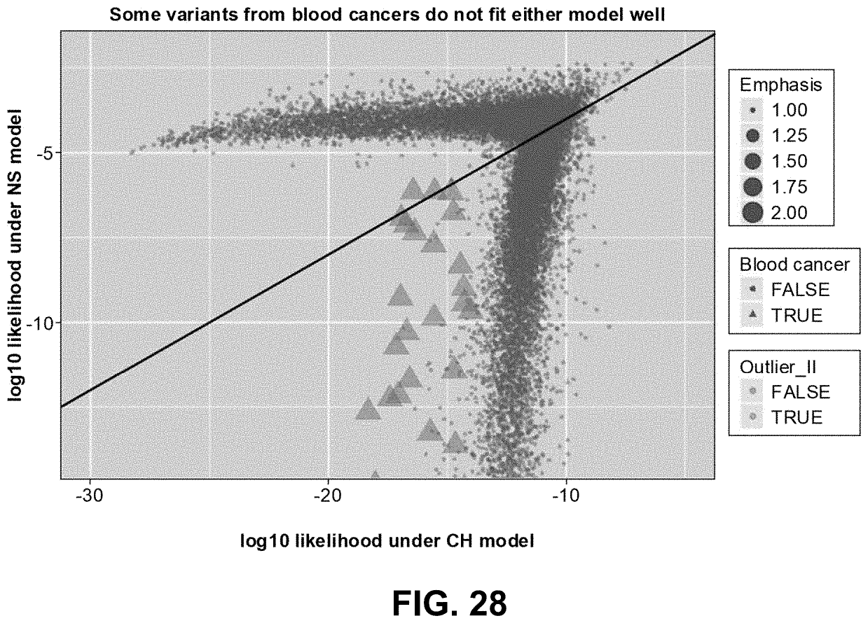

FIG. 28 illustrates detected outliers of a distribution of training data sets according to some embodiments. In some embodiments, the mixture model 225 can classify a candidate variant as outliers responsive to determining that the candidate variant does not fit with the distributions for either clonal hematopoiesis or novel somatic variants. For instance, the mixture model 225 determines that each of the likelihoods of belonging to one of the sources is less than a threshold likelihood, which can occur more frequently for certain genes associated with greater average noise or lower average signal in sequencing data. In the example shown in FIG. 28, data points for blood-related cancers can be classified as outliers because they do not resemble data points from age-related clonal hematopoiesis or novel somatic cancers.

### VIII. Computing Machine Architecture

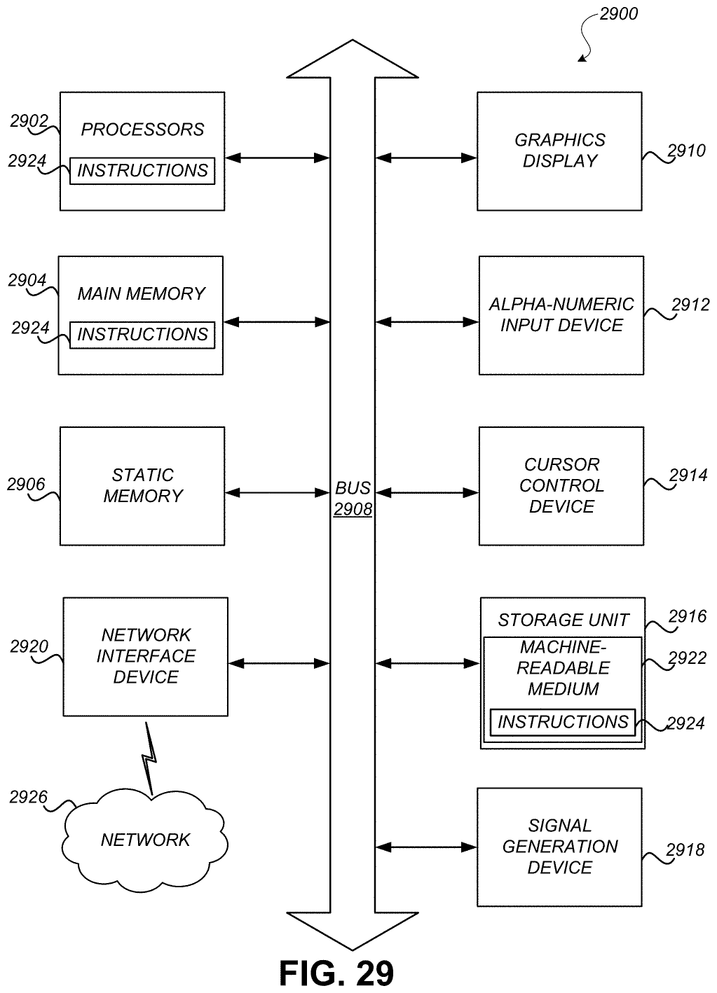

FIG. 29 shows a schematic of an example computer system for implementing various methods of the present invention. In particular, FIG. 29 is a block diagram illustrating components of an example computing machine that is capable of reading instructions from a computer-readable medium and execute them in a processor (or controller). A computer described herein may include a single computing machine shown in FIG. 29, a virtual machine, a distributed computing system that includes multiples nodes of computing machines shown in FIG. 29, or any other suitable arrangement of computing devices.

By way of example, FIG. 29 shows a diagrammatic representation of a computing machine in the example form of a computer system 2900 within which instructions 2924 (e.g., software, program code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 29 may correspond to any software, hardware, or combined components (e.g., those shown in FIG. 2 or a processing unit described herein), including but not limited to any engines, modules, computing server, machines that are used to perform one or more processes described herein. While FIG. 29 shows various hardware and software elements, each of the components described herein may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 2924 that specify actions to be taken by that machine. Further, while only a single machine is illustrated, the term “machine” and “computer” may also be taken to include any collection of machines that individually or jointly execute instructions 2924 to perform any one or more of the methodologies discussed herein.

The example computer system 2900 includes one or more processors 2902 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 2900 may also include a memory 2904 that store computer code including instructions 2924 that may cause the processors 2902 to perform certain actions when the instructions are executed, directly or indirectly by the processors 2902. Instructions can be any directions, commands, or orders that may be stored in different forms, such as equipment-readable instructions, programming instructions including source code, and other communication signals and orders. Instructions may be used in a general sense and are not limited to machine-readable codes.

One and more methods described herein improve the operation speed of the processors 2902 and reduces the space required for the memory 2904. For example, the machine learning methods described herein reduces the complexity of the computation of the processors 2902 by applying one or more novel techniques that simplify the steps in training, reaching convergence, and generating results of the processors 2902. The algorithms described herein also reduces the size of the models and datasets to reduce the storage space requirement for memory 2904.

The performance of certain of the operations may be distributed among the more than processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations. Even though in the specification or the claims may refer some processes to be performed by a processor, this should be construed to include a joint operation of multiple distributed processors.

The computer system 2900 may include a main memory 2904, and a static memory 2906, which are configured to communicate with each other via a bus 2908. The computer system 2900 may further include a graphics display unit 2910 (e.g., a plasma display panel (PDP), a liquid crystal display (LCD), a projector, or a cathode ray tube (CRT)). The graphics display unit 2910, controlled by the processors 2902, displays a graphical user interface (GUI) to display one or more results and data generated by the processes described herein. The computer system 2900 may also include alphanumeric input device 2912 (e.g., a keyboard), a cursor control device 2914 (e.g., a mouse, a trackball, a joystick, a motion sensor, or other pointing instrument), a storage unit 2916 (a hard drive, a solid state drive, a hybrid drive, a memory disk, etc.), a signal generation device 2918 (e.g., a speaker), and a network interface device 2920, which also are configured to communicate via the bus 2908.

The storage unit 2916 includes a computer-readable medium 2922 on which is stored instructions 2924 embodying any one or more of the methodologies or functions described herein. The instructions 2924 may also reside, completely or at least partially, within the main memory 2904 or within the processor 2902 (e.g., within a processor's cache memory) during execution thereof by the computer system 2900, the main memory 2904 and the processor 2902 also constituting computer-readable media. The instructions 2924 may be transmitted or received over a network 2926 via the network interface device 2920.

While computer-readable medium 2922 is shown in an example embodiment to be a single medium, the term “computer-readable medium” should be taken to include a single medium or multiple media (e.g., a centralized or distributed database, or associated caches and servers) able to store instructions (e.g., instructions 2924). The computer-readable medium may include any medium that is capable of storing instructions (e.g., instructions 2924) for execution by the processors (e.g., processors 2902) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### IX. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

1. A method for determining a source of a variant in a cell free nucleic acid sample, the method comprising:
identifying a candidate variant in the cell free nucleic acid sample;
determining a numerical score using a measure of first properties of a distribution of novel somatic mutations compared to a measure of second properties of a distribution of somatic variants matched in genomic nucleic acid; and
determining a classification of the candidate variant using the numerical score, the classification indicating whether the candidate variant is more likely to be a new novel somatic mutation than a new somatic variant matched in genomic nucleic acid.

2. The method of claim 1, wherein the first properties of the distribution of novel somatic mutations and the second properties of the distribution of somatic variants matched in genomic nucleic acid are modeled by generalized linear models.

3. The method of claim 2, wherein the generalized linear models each generate outcomes from a gamma distribution.

4. The method of claim 2, wherein the generalized linear models each generate outcomes from a normal distribution, binomial distribution, or Poisson distribution.

5. The method of claim 2, wherein the generalized linear models are trained by modeling a true alternate frequency of the candidate variant in a given genomic nucleic acid sample as dependent on a true alternate frequency of the candidate variant in a given cell free nucleic acid sample.

6. The method of any one of claim 1, wherein the numerical score is determined at least by modeling alternate allele counts of the candidate variant using a Poisson distribution after a gamma distribution.

7. The method of claim 1, wherein the measure of first properties or the measure of second properties represents a likelihood under a generalized linear model using a gamma distribution with Poisson counts.

8. The method of claim 1, wherein the first and second properties include one or more of: depth, alternate frequency, or trinucleotide context of a given nucleic acid sample.

9. The method of claim 1, wherein the numerical score is further determined by comparing the first properties, the second properties, and third properties of a distribution of variants associated with a source different from the novel somatic mutations and the somatic variants matched in genomic nucleic acid.

10. The method of claim 1, wherein the somatic variants matched in genomic nucleic acid are matched with variants observed in white blood cells.

11. The method of claim 1, wherein determining the numerical score comprises:
determining a first likelihood l_{NS} of observing the novel somatic mutations based on an alternate frequency of the novel somatic mutations.

12. The method of claim 11, further comprising:
determining that the candidate variant is located on a certain gene; and
determining a second likelihood π_{NS,gene} that the certain gene will have at least one mutation based on observed data from training samples of the certain gene,
wherein the numerical score is determined based at least in part on a product of the first likelihood and the second likelihood.

13. The method of claim 12, further comprising:
determining an attribute of an individual from whom the cell free nucleic acid sample was obtained; and
determining a third likelihood π_{NS,person} that the individual will have the candidate variant based on observed data from training samples of individuals associated with the attribute, the product further including the third likelihood.

14. The method of claim 13, wherein the attribute is an age or an age range.

15. The method of claim 1, wherein determining the classification of the candidate variant comprises:
determining an integral of a plurality of negative binomial distributions over an expected distribution of alternate frequency of the candidate variant in a given cell free nucleic acid sample.

16. The method of claim 15, wherein the plurality of negative binomial distributions model expected distributions of false positive and true positive mutations of the candidate variant in a given cell free nucleic acid sample.

17. The method of claim 15, wherein the plurality of negative binomial distributions account for depths of sequence reads of samples of the novel somatic mutations and the somatic variants matched in genomic nucleic acid.

18. The method of claim 1, wherein the somatic variants matched in genomic nucleic acid are associated with clonal hematopoiesis.

19. The method of claim 1, further comprising:
determining a prediction that the candidate variant is a true mutation in the cell free nucleic acid sample based on the classification; and
determining a likelihood that an individual has a disease based at least in part on the prediction.

20. A method for modeling sources of variants in nucleic acid samples, the method comprising:
obtaining a first set of training samples of novel somatic mutations;
obtaining a second set of training samples of somatic variants matched in genomic nucleic acid;
determining a first shape parameter and a first slope parameter of a first generalized linear model by iteratively modeling variance of the first set of training samples as a first gamma distribution;
determining a second shape parameter and a second slope parameter of a second generalized linear model by iteratively modeling variance of the second set of training samples as a second gamma distribution; and
storing the first and second shape parameters and the first and second slope parameters, the stored parameters used for determining whether a candidate variant is more likely to be a novel somatic mutation than a somatic variant matched in genomic nucleic acid.

21. The method of claim 20, wherein iteratively modeling variance of the first and second sets of training samples comprises:
modifying at least one of the first and second sets of training samples using samples of individuals known to have a certain disease or not;
determining updated parameters for the first and second generalized linear models using the modified at least one training sample; and
determining pairs of precision and recall values for predicting true mutations of a test set of cell free nucleic acid samples using the updated parameters.

22. The method of claim 21, further comprising:
determining a precision value and a corresponding recall value of one of the pairs of precision and recall values;
determining that the precision value is greater than a threshold precision; and
determining that the recall value is greater than a threshold recall.

23. The method of claim 21, wherein the updated parameters are iteratively determined using an expectation-maximization algorithm.

24. A system comprising a computer processor and a memory, the memory storing computer program instructions that when executed by the computer processor cause the processor to:
identify a candidate variant in a cell free nucleic acid sample;
determine a numerical score using a measure of first properties of a distribution of novel somatic mutations compared to a measure of second properties of a distribution of somatic variants matched in genomic nucleic acid; and
determine a classification of the candidate variant using the numerical score, the classification indicating whether the candidate variant is more likely to be a new novel somatic mutation than a new somatic variant matched in genomic nucleic acid.
